(19) United States
(12) Patent Application Publication
(10) Pub. No.: US 2005/0232918 A1
(43) Pub. Date: Oct. 20, 2005

(54) Differentially expressed genes associated with obesity and type 2 diabetes

(76) Inventors: Greg Collier, Victoria (AU); Paul Zev Zimmet, Victoria (AU); Ken Walder, Victoria (AU); Janine McMillan, Victoria (AU); Andrea Michelle Sanigorski, Victoria (AU); Kelly Windmill, Victoria (AU)

Correspondence Address: The McCallum Law Firm, LLC., 132 Kolar Ct., Erie, CO, 80516, US

(21) Appl. No.: 10/826,448

(22) Filed: Apr. 16, 2004

(51) Int. Cl. C12Q 1/68; C07H 21/04; A61K 39/395; C07K 14/705; C07K 16/28; C12N 15/09

(52) U.S. Cl. 424/143.1; 435/6; 435/69.1; 435/320.1; 435/325; 530/350; 530/388.22; 536/23.5

(57) ABSTRACT

The present invention relates generally to a nucleic acid molecule which is expressed in at least the stomach, hypothalamus or liver identified using differential display techniques under differing physiological conditions. It is proposed that the nucleic acid molecules encode expression products associated with the modulation of obesity, anorexia, weight maintenance, diabetes and/or metabolic energy levels. More particularly, the present invention is directed to nucleic acid molecules and expression products produced by recombinant means from the nucleic acid molecule or isolated from natural sources and their use in therapeutic and diagnostic protocols for conditions such as obesity, anorexia, weight maintenance, diabetes and/or energy imbalance. The subject nucleic acid molecule and expression products and their derivatives, homologs, analogs and mimetics are proposed to be useful, therefore, as therapeutic and diagnostic agents for obesity, anorexia, weight maintenance, diabetes and/or energy imbalance or as targets for the design and/or identification of modulators of their activity and/or function.

### REFERENCE TO RELATED APPLICATIONS

This is a United States Utility patent application claiming priority to U.S. Provisional Application Ser. No. 60/330,149 filed Oct. 16, 2001, which was later converted to PCT Application Ser. No. PCT/AU02/001405 filed Oct. 16, 2002. Both applications are herein incorporated by reference in their entirety.

### FIELD OF THE INVENTION

The present invention relates generally to a nucleic acid molecule which is expressed in at least the stomach, hypothalamus or liver identified using differential display techniques under differing physiological conditions. It is proposed that the nucleic acid molecules encode expression products associated with the modulation of obesity, anorexia, weight maintenance, diabetes and/or metabolic energy levels. More particularly, the present invention is directed to nucleic acid molecules and expression products produced by recombinant means from the nucleic acid molecule or isolated from natural sources and their use in therapeutic and diagnostic protocols for conditions such as obesity, anorexia, weight maintenance, diabetes and/or energy imbalance. The subject nucleic acid molecule and expression products and their derivatives, homologs, analogs and mimetics are proposed to be useful, therefore, as therapeutic and diagnostic agents for obesity, anorexia, weight maintenance, diabetes and/or energy imbalance or as targets for the design and/or identification of modulators of their activity and/or function.

### BACKGROUND OF THE INVENTION

Bibliographic details of references provided in the subject specification are listed at the end of the specification.

The increasing sophistication of recombinant DNA technology is greatly facilitating research and development in the medical, veterinary and allied human and animal health fields. This is particularly the case in the investigation of the genetic bases involved in the etiology of certain disease conditions. One particularly significant condition from the stand point of morbidity and mortality is obesity and its association with type 2 diabetes (formerly non-insulin-dependent diabetes mellitus or NIDDM) and cardiovascular disease.

Obesity is defined as a pathological excess of body fat and is the result of an imbalance between energy intake and energy expenditure for a sustained period of time. Obesity is the most common metabolic disease found in affluent nations. The prevalence of obesity in these nations is alarmingly high, ranging from 10% to upwards of 50% in some subpopulations (Bouchard, The genetics of obesity. Boca Raton: CRC Press, 1994). Of particular concern is the fact that the prevalence of obesity appears to be rising consistently in affluent societies and is now increasing rapidly in less prosperous nations as they become more affluent and/or adopt cultural practices from the more affluent countries (Zimmet, Diabetes Care 15(2): 232-247, 1992).

In 1995 in Australia, for example, 19% of the adult population were obese (BMI>30). On average, women in 1995 weighed 4.8 kg more than their counterparts in 1980 while men weighed 3.6 kg more (Australian Institute of Health and Welfare, Heart, Stroke and Vascular diseases, Australian facts. AIHW Cat. No. CVD 7 Canberra: AIHW and the Heart Foundation of Australia, 1999). More recently, the AusDiab Study conducted between the years 1999 and 2000 showed that 65% of males and 45% of females aged 25-64 years were considered overweight (de Looper and Bhatia, Australia's Health Trends 2001. Australian Institute of Health and Welfare (AIHW) Cat. No. PHE 24. Canberra: AIHW, 2001). The prevalence of obesity in the United States also increased substantially between 1991 and 1998, rising from 12% to 18% in Americans during this period (Mokdad et al., JAMA. 282(16): 1519-1522, 1999).

The high and increasing prevalence of obesity has serious health implications for both individuals and society as a whole. Obesity is a complex and heterogeneous disorder and has been identified as a key risk indicator of preventable morbidity and mortality since obesity increases the risk of a number of other metabolic conditions including type 2 diabetes mellitus and cardiovascular disease (Must et al., JAMA. 282(16): 1523-1529, 1999; Kopelman, Nature 404: 635-643, 2000). Alongside obesity, the prevalence of diabetes continues to increase rapidly. It has been estimated that there were about 700,000 persons with diabetes in Australia in 1995 while in the US, diabetes prevalence increased from 4.9% in 1990 to 6.9% in 1999 (Mokdad, Diabetes Care 24(2): 412, 2001). In Australia, the annual costs of obesity associated with diabetes and other disease conditions has been conservatively estimated to be AU$810 million for 1992-3 (National Health and Medical Research Council, Acting on Australia's weight: A strategy for the prevention of overweight and obesity. Canberra: National Health and Medical Research Council, 1996). In the U.S., the National Health Interview Survey (NHIS) estimated the economic cost of obesity in 1995 as approximately US$99 billion, thereby representing 5.7% of total health costs in the U.S. at that time (Wolf and Colditz, Obes Res. 6: 97-106, 1998).

A genetic basis for the etiology of obesity is indicated inter alia from studies in twins, adoption studies and population-based analyses which suggest that genetic effects account for 25-80% of the variation in body weight in the general population (Bouchard, 1994, supra; Kopelman et al., Int J Obesity 18: 188-191, 1994; Ravussin, Metabolism 44(Suppl 3): 12-14, 1995). It is considered that genes determine the possible range of body weight in an individual and then the environment influences the point within this range where the individual is located at any given time (Bouchard, 1994, supra). However, despite numerous studies into genes thought to be involved in the pathogenesis of obesity, there have been surprisingly few significant findings in this area. In addition, genome-wide scans in various population groups have not produced definitive evidence of the chromosomal regions having a major effect on obesity.

A number of organs/tissues have been implicated in the pathophysiology of obesity and type 2 diabetes, and of particular interest are the hypothalamus, stomach and liver. The hypothalamus has long been recognized as a key brain area in the regulation of energy intake (Stellar, Psychol Rev 61: 5-22, 1954) and it is now widely accepted that the hypothalamus plays a central role in energy homeostasis, integrating and co-ordinating a large number of factors produced by and/or acting on the hypothalamus. A number of these factors have been investigated for their role in energy balance and body weight regulation, including neuropeptide Y, corticotropin-releasing hormone, melanin-concentrating hormone, leptin and insulin. It has been proposed that genetic alterations perturbing the metabolic pathways regulating energy balance in the hypothalamus could contribute to the development of obesity, and subsequently diabetes. Thus, an important step in understanding the function of the hypothalamus in regulating the metabolism of an animal requires the identification of the targets of these hormones. Such targets may be whole organs, and genes whose expression is regulated by the presence of these hormones.

The role of the stomach in regulating food intake is thought to involve two types of signals: the degree of distension of the stomach and the activation of chemoreceptors in the gastric or intestinal wall (Koopmans, Experimental studies on the control of food intake. In: Handbook of Obesity, Eds. GA Bray, C Bouchard, WPT James pp 273-312, 1998). The gut is the largest endocrine organ in the body and after a meal a large number of gastrointestinal hormones are released. Some examples are gastrin, somatostatin, cholecystokinin, gastric inhibitory polypeptide and neurotensin. Despite general agreement that the stomach provides part of the signal that restricts food intake during a single meal, the nature of this signal or how it is transmitted to the brain remains to be determined. Most likely the information relating to the degree of distension of the stomach or the presence of nutrients in the gastrointestinal walls is transmitted to the brain through either nerves or hormones. The role of the gut hormones identified to date in the regulation of food intake remains to be equivocally determined.

The liver also plays a significant role in a number of important physiological pathways. It has a major role in the regulation of metabolism of glucose, amino acids and fat. In addition the liver is the only organ (other than the gut) that comes into direct contact with a large volume of ingested, absorbed food via the portal vein and, therefore, the liver is able to “sense” or monitor the level of nutrients entering the body, particularly the amounts of protein and carbohydrate. It has been proposed that the liver may also have a role in the regulation of food intake through the transmission of unidentified signals relaying information to the brain about nutrient absorption from the gut and metabolic changes throughout the body (Russek, Nature 200: 176, 1963). The liver also plays a crucial role in maintaining circulating glucose concentrations by regulating pathways such as gluconeogenesis and glycogenolysis. Alterations in glucose homeostasis are important factors in the pathophysiology of impaired glucose tolerance and the development of type 2 diabetes mellitus.

In accordance with the present invention, genetic sequences were sought which are differentially expressed in particular vertebrate animal tissues or organs between either fed, re-fed or fasting conditions, or between diabetic and non-diabetic conditions. Novel genes are identified which are differentially expressed at least in the stomach, liver and/or hypothalamus under one or both of the above-mentioned conditions. In accordance with the present invention, the inventors have isolated genes which are proposed to be associated with one or more biological functions associated with disease conditions such as but not limited to obesity, anorexia, diabetes or energy balance.

### SUMMARY OF THE INVENTION

Analysis of genetic material from stomach, hypothalamus and liver tissue were used to identify candidate genetic sequences associated with a healthy state or with physiological conditions such as obesity, anorexia, weight maintenance, diabetes and/or metabolic energy levels. An animal model was employed comprising the Israeli Sand Rat (Psammomys obesus). Three groups of animals were used designated Groups A, B and C based on metabolic phenotype as follows:—
Group A: lean animals (normoglycemic; normoinsulinemic);
Group B: obese, non-diabetic animals (normoglycemic; hyperinsulinemic); and
Group C: obese, diabetic animals (hyperglycemic; hyperinsulinemic).

Techniques including differential display PCR analysis, suppression substractive hybridization (SSH) and amplified fragment length polymorphism analysis of mRNA from stomach, liver or hypothalamus tissue were used to identify genetic sequences differentially expressed in fed, re-fed and fasted mammals or in diabetic and non-diabetic mammals. The Israeli Sand Rat (Psammomys obesus) was found to be particularly useful for this analysis. In a particular embodiment, seven differentially expressed sequences were identified designated herein AGT-119 [SEQ ID NO:1], AGT-120 [SEQ ID NO:2], AGT-121 [SEQ ID NO:3], AGT-122 [SEQ ID NO:5], AGT-422 [SEQ ID NO:6], AGT-123 [SEQ ID NO:7] and AGT-504 [SEQ ID NO:8 and SEQ ID NO:9]. SEQ ID NO:9 is a genomic form of AGT-504 and is also represented as SEQ ID NO:8.

AGT-119 was detected initially in stomach tissue using differential display PCR and its expression was elevated in fed animals compared to fasted or re-fed animals. AGT-120 was initially detected in stomach tissue using differential display PCR and its expression was elevated in fed animals compared to fasted or re-fed animals. AGT-121 was initially identified in the hypothalamus using differential display PCR and its expression levels were elevated in fasted diabetic, non-diabetic and lean animals compared to fed animals when separated by genotype. AGT-122 was initially identified in the liver using differential display PCR and was shown to have elevated expression levels in fasted compared to fed diabetic or non-diabetic animals. AGT-422 was identified suppression subtractive hybridization in liver tissue and its expression was elevated in fed lean animals compared to fed diabetic animals and elevated in fed, lean animals compared to fasted lean animals, in fed non-diabetic animals compared to fasted non-diabetic animals and fed diabetic animals compared to fasted diabetic animals. AGT-123 was identified in the hypothalamus tissue using differential display PCR and its expression was found to be elevated in fasted lean, non-diabetic animals and diabetic animals compared to fed animals. AGT-504 was identified using amplified fragment length polymorphism analysis in genomic DNA and its expression in liver tissue was elevated in diabetic animals compared to lean or non-diabetic animals. A summary of the AGT genes is provided in Table 1.

TABLE 1
Summary of Differentially Expressed Genes

| | SEQ ID | | | DETECTION |
|---|---|---|---|---|
| GENE | NO: | TISSUE | CHARACTERISTICS | METHOD |
| AGT-119 | 1 | stomach | elevated expression in | differential |
| | | | fed animals compared to | display PCR |
| | | | fasted or re-fed animals | |
| AGT-120 | 2 | stomach | elevated expression in | differential |
| | | | fed animals compared to | display PCR |
| | | | fasted or re-fed animals | |
| AGT-121 | 3 and 4 | hypothalamus | elevated expression in | differential |
| | | | fasted diabetic, non- | display PCR |
| | | | diabetic and lean animals | |
| | | | compared to fed animals | |
| | | | when separated by | |
| | | | genotype | |
| AGT-122 | 5 | liver | elevated expression in | differential |
| | | | fasted compared to fed | display PCR |
| | | | diabetic and non-diabetic | |
| | | | animals | |
| AGT-422 | 6 | liver | elevated expression in | suppression |
| | | | fed lean animals | subtractive |
| | | | compared to fed diabetic | hybridization |
| | | | animals and elevated | (representational |
| | | | expression in fed lean | difference |
| | | | animals compared to | analysis) |
| | | | fasted lean animals, in | |
| | | | fed non-diabetic animals | |
| | | | compared to fasted non- | |
| | | | diabetic animals and fed | |
| | | | diabetic animals compared | |
| | | | to fasted diabetic animals | |
| AGT-123 | 7 | hypothalamus | elevated expression in | differential |
| | | | fasted lean, non-diabetic | display PCR |
| | | | and diabetic animals | |
| | | | compared to fed animals | |
| AGT-504 | 8 | liver | elevated expression in | amplified |
| | (genomic) | | diabetic animals | fragment length |
| | and 9 | | compared to lean or non- | polymorphism |
| | (cDNA) | | diabetic animals | analysis |

The present invention provides, therefore, a nucleic acid molecule comprising a sequence of nucleotides encoding or complementary to a sequence encoding an expression product or a derivative, homolog, analog or mimetic thereof wherein said nucleic acid molecule is differentially expressed in one or more of stomach, liver or hypothalamus tissue under fed or unfed or diabetic or non-diabetic conditions.

The present invention further provides a nucleic acid molecule comprising a nucleotide sequence encoding or complementary to a sequence encoding an expression product or a derivative, homolog, analog or mimetic thereof wherein the nucleotide sequence is as substantially set forth in SEQ ID NO:1, SEQ ID NO:2, SEQ ID NO:3, SEQ ID NO:5, SEQ ID NO:6, SEQ ID NO:7, SEQ ID NO:8 or SEQ ID NO:9 or a nucleotide sequence having at least about 30% similarity to all or part of SEQ ID NO:1, SEQ ID NO:2, SEQ ID NO:3, SEQ ID NO:4, SEQ ID NO:5, SEQ ID NO:6, SEQ ID NO:7, SEQ ID NO:8 or SEQ ID NO:9 and/or is capable of hybridizing to one or more of SEQ ID NO:1, SEQ ID NO:2, SEQ ID NO:3, SEQ ID NO:4, SEQ ID NO:5, SEQ ID NO:6, SEQ ID NO:7, SEQ ID NO:8 or SEQ ID NO:9 or their complementary forms under low stringency conditions at 42° C. and wherein the nucleic acid molecule is differentially expressed in one or more of stomach, liver or hypothalamus tissue under fed or unfed or diabetic or non-diabetic conditions.

The present invention also provides an isolated expression product or a derivative, homolog, analog or mimetic thereof which expression product is encoded by a nucleotide sequence which is differentially expressed in one or more of stomach, liver or hypothalamus tissue under fed or unfed or diabetic or non-diabetic conditions.

More particularly, the present invention is directed to an isolated expression product or a derivative, homolog, analog or mimetic thereof wherein the expression product is encoded by a nucleotide sequence substantially as set forth in SEQ ID NO:1, SEQ ID NO:2, SEQ ID NO:3, SEQ ID NO:5, SEQ ID NO:6, SEQ ID NO:7, SEQ ID NO:8 or SEQ ID NO:9 or a nucleotide sequence having at least 30% similarity to all or part of SEQ ID NO:1, SEQ ID NO:2, SEQ ID NO:3, SEQ ID NO:5, SEQ ID NO:6, SEQ ID NO:7, SEQ ID NO:8 or SEQ ID NO:9 and/or is capable of hybridizing to SEQ ID NO:1, SEQ ID NO:2, SEQ ID NO:3, SEQ ID NO:5, SEQ ID NO:6, SEQ ID NO:7, SEQ ID NO:8 or SEQ ID NO:9 or their complementary forms under low stringency conditions at 42° C.

The genetic sequence of the present invention may be referred to herein as AGT-119, AGT-120, AGT-121, AGT-122, AGT-422, AGT-123 and AGT-504. The expression products encoded by AGT-119, AGT-120, AGT-121, AGT-122, AGT-422, AGT-123 and AGT-504 may be referred to herein as AGT-119, AGT-120, AGT-121, AGT-122, AGT-422, AGT-123 and AGT-504, respectively. The expression product may be an RNA (e.g. mRNA) or a protein. Where the expression product is an RNA, the present invention extends to RNA-related molecules associated thereto such as RNAi or intron or exon sequences therefrom.

Even yet another aspect of the present invention relates to a composition comprising AGT-119, AGT-120, AGT-121, AGT-122, AGT-422, AGT-123 and/or AGT-504 or its derivatives, homologs, analogs or mimetics or agonists or antagonists of AGT-119, AGT-120, AGT-121, AGT-122, AGT-422, AGT-123 and/or AGT-504 together with one or more pharmaceutically acceptable carriers and/or diluents.

Another aspect of the present invention contemplates a method for treating a subject comprising administering to said subject a treatment effective amount of AGT-119, AGT-120, AGT-121, AGT-122, AGT-422, AGT-123 and/or AGT-504 or a derivative, homolog, analog or mimetic thereof or a genetic sequence encoding same or an agonist or antagonist of AGT-119, AGT-120, AGT-121, AGT-122, AGT-422, AGT-123 and/or AGT-504 activity or AGT-119, AGT-120, AGT-121, AGT-122, AGT-422, AGT-123 and/or AGT-504 gene expression for a time and under conditions sufficient to effect treatment.

In accordance with this and other aspects of the present invention, treatments contemplated herein include but are not limited to obesity, anorexia, weight maintenance, energy imbalance and diabetes. Treatment may be by the administration of a pharmaceutical composition or genetic sequences via gene therapy. Treatment is contemplated for human subjects as well as animals such as animals important to the livestock industry.

A further aspect of the present invention is directed to a diagnostic agent for use in monitoring or diagnosing conditions such as but not limited to obesity, anorexia, weight maintenance, energy imbalance and/or diabetes, said diagnostic agent selected from an antibody to AGT-119, AGT-120, AGT-121, AGT-122, AGT-422, AGT-123 or AGT-504 or its derivatives, homologs, analogs or mimetics and a genetic sequence comprising or capable of annealing to a nucleotide strand associated with AGT-119, AGT-120, AGT-121, AGT-122, AGT-422, AGT-123 or AGT-504 useful inter alia in a diagnostic procedure such as PCR, hybridization, RFLP analysis or AFLP analysis and the like.

A summary of sequence identifiers used throughout the subject specification is provided in Table 2.

TABLE 2

| SEQUENCE ID NO: | DESCRIPTION |
|---|---|
| 1 | Nucleotide sequence of AGT-119 |
| 2 | Nucleotide sequence of AGT-120 |
| 3 | Nucleotide sequence of AGT-121 |
| 4 | Corresponding amino acid of SEQ ID NO: 3 |
| 5 | Nucleotide sequence of AGT-122 |
| 6 | Nucleotide sequence of AGT-422 |
| 7 | Nucleotide sequence of AGT-123 |
| 8 | Nucleotide sequence of AGT-504 (genomic) |
| 9 | Nucleotide sequence of AGT-504 (cDNA) |
| 10 | primer |
| 11 | primer |
| 12 | AGT-119 (set 1) forward primer |
| 13 | AGT-119 (set 1) reverse primer |
| 14 | AGT-119 (set 2) forward primer |
| 15 | AGT-119 (set 2) reverse primer |
| 16 | AGT-120 forward primer |
| 17 | AGT-120 reverse primer |
| 18 | AGT-121 forward (insertion) primer |
| 19 | AGT-121 forward (deletion) primer |
| 20 | AGT-121 reverse primer |
| 21 | AGT-122 forward primer |
| 22 | AGT-122 reverse primer |
| 23 | AGT-422 forward primer |
| 24 | AGT-422 reverse primer |
| 25 | AGT-123 forward primer |
| 26 | AGT-123 reverse primer |
| 27 | AGT-504 forward primer |
| 28 | AGT-504 reverse primer |
| 29 | β-actin forward primer |
| 30 | β-actin reverse primer |
| 31 | β-actin probe |
| 32 | Cyclophilin forward primer |
| 33 | Cyclophilin reverse primer |
| 34 | Cyclophilin probe |

### BRIEF DESCRIPTION OF THE DRAWINGS

FIG. 1 illustrates a graphical representation of AGT-119 stomach gene expression in fed, fasted and re-fed Psammomys obesus according to an embodiment of the present invention.

FIG. 2 illustrates a graphical representation of AGT-120 stomach gene expression in fasted, fed and re-fed Psammomys obesus according to another embodiment of the present invention.

FIG. 3 illustrates a graphical representation of the distribution of tissue in which AGT-121 is expressed according to an embodiment of the present invention.

FIG. 4 illustrates a photographic representation of Northern analysis of AGT-121 expression in (1) heart; (2) brain; (3) placenta; (4) lung; (5) liver; (6) skeletal muscle); (7) kidney and (8) pancreas according to an embodiment of the present invention.

FIG. 5 illustrates a graphical representation of AGT-121 expression in energy restricted hypothalamus according to an embodiment of the present invention.

FIG. 6 illustrates a graphical representation of expression of AGT-121 versus level of body weight according to an embodiment of the present invention.

FIG. 7 illustrates a graphical representation of expression of AGT-121 versus change in glucose levels according to an embodiment of the present invention.

FIG. 8 illustrates a graphical representation of expression of AGT-121 versus scap fat according to an embodiment of the present invention.

FIG. 9 illustrates a graphical representation of AGT-122 hepatic gene expression in Groups A, B and C fed and fasted Psammomys obesus according to an embodiment of the present invention.

FIG. 10 illustrates a graphical representation of AGT-122 hepatic gene expression for fed and fasted Psammomys obesus (pooled data) according to an embodiment of the present invention.

FIG. 11 illustrates a graphical representation of the association between AGT-122 hepatic gene expression and body weight in fed Psammomys obesus according to an embodiment of the present invention.

FIG. 12 illustrates a graphical representation of AGT-422 hepatic gene expression data from Groups A, B and C fed and fasted Psammomys obesus according to an embodiment of the present invention.

FIG. 13 illustrates a graphical representation of AGT-422 hepatic gene expression in all fed and fasted Psammomys obesus (pooled data) according to an embodiment of the present invention.

FIG. 14 illustrates a graphical representation of AGT-123 hypothalamic gene expression in Groups A, B and C fed and fasted Psammomys obesus according to an embodiment of the present invention.

FIG. 15 illustrates a graphical representation of AGT-504 hepatic gene expression in Groups A, B and C Psammomys obesus according to an embodiment of the present invention.

### DETAILED DESCRIPTION OF THE INVENTION

The present invention is predicated in part on the identification of novel genes associated inter alia with regulation of obesity, anorexia, weight maintenance, diabetes and/or metabolic energy levels. The genes were identified following differential screening of mRNA from one or more of stomach, liver or hypothalamus tissue in fed, re-fed and fasted mammals or in diabetic and non-diabetic mammals. The selection of stomach, liver and hypothalamus is not intended to imply that differential expression does not occur in other tissue.

Accordingly, one aspect of the present invention provides a nucleic acid molecule comprising a sequence of nucleotides encoding or complementary to a sequence encoding an expression product or a derivative, homolog, analog or mimetic thereof wherein said nucleic acid molecule is differentially expressed in one or more of stomach, liver or hypothalamus tissue under fed (or re-fed) or unfed or diabetic or non-diabetic conditions.

The term “differentially expressed” is used in its most general sense and includes elevated levels of an expression product such as mRNA or protein or a secondary product such as cDNA in one tissue compared to another tissue or in the same tissue but under different conditions. Examples of different conditions include differential expression in tissue from fed, re-fed and fasting animals or diabetic and non-diabetic animals. Differential expression is conveniently determined by a range of techniques including polymerase chain reaction (PCR) such as real-time PCR. Other techniques include suppression subtractive hyridization (SSH), amplified fragment length polymorphism (AFLP) analysis, and the like.

Conveniently, an animal model may be employed to study the differences in gene expression in animal tissues under different conditions. In particular, the present invention is exemplified using the Psammomys obesus (the Israeli Sand Rat) animal model of dietary-induced obesity and type 2 diabetes. In their natural desert habitat, an active lifestyle and saltbush diet ensure that they remain lean and normoglycemic (Shafrir and Gutman, J. Basic Clin. Physiol. Pharm. 4: 83-99, 1993). However, in a laboratory setting on a diet of ad libitum chow (on which many other animal species remain healthy), a range of pathophysiological responses are seen (Barnett et al., Diabetologia 37: 671-676, 1994a; Barnett et al., Int. J. Obesity 18: 789-794, 1994b; Barnett et al., Diabete Nutr. Metab. 8: 42-47, 1995). By the age of 16 weeks, more than half of the animals become obese and approximately one third develop type 2 diabetes. Only hyperphagic animals go on to develop hyperglycemia, highlighting the importance of excessive energy intake in the pathophysiology of obesity and type 2 diabetes in Psammomys obesus (Collier et al., Ann. New York Acad. Sci. 827: 50-63, 1997a; Walder et al., Obesity Res. 5: 193-200, 1997a). Other phenotypes found include hyperinsulinemia, dyslipidemia and impaired glucose tolerance (Collier et al., 1997a, supra; Collier et al., Exp. Clin. Endocrinol. Diabetes 105: 36-37, 1997b). Psammomys obesus exhibit a range of bodyweight and blood glucose and insulin levels which form a continuous curve that closely resembles the patterns found in human populations, including the inverted U-shaped relationship between blood glucose and insulin levels known as “Starling's curve of the pancreas” (Barnett et al., 1994a, supra). It is the heterogeneity of the phenotypic response of Psammomys obesus which makes it an ideal model to study the etiology and pathophysiology of obesity and type 2 diabetes.

The animals are conveniently classified into three groups designated Groups A, B and C:
Group A: animals are lean;
Group B: animals are obese and non-diabetic; and
Group C: animals are obese and diabetic.

In accordance with the present invention, a number of differentially expressed genetic sequences were identified in stomach, liver or hypothalamus tissue in Psammomys obesus under different feeding regimes (i.e. fed, re-fed or fasting) or under diabetic or non-diabetic conditions.

Another aspect of the present invention provides a nucleic acid molecule comprising a nucleotide sequence encoding or complementary to a sequence encoding an expression product or a derivative, homolog, analog or mimetic thereof wherein said nucleotide sequence is as substantially set forth in SEQ ID NO:1 (AGT-119), SEQ ID NO:2 (AGT-120), SEQ ID NO:3 (AGT-121), SEQ ID NO:5 (AGT-122), SEQ ID NO:6 (AGT-422), SEQ ID NO:7 (AGT-123), SEQ ID NO:8 (AGT-504 genomic) or SEQ ID NO:9 (AGT-504 cDNA) or a nucleotide sequence having at least about 30% similarity to all or part of SEQ ID NO:1, SEQ ID NO:2, SEQ ID NO:3, SEQ ID NO:5, SEQ ID NO:6, SEQ ID NO:7, SEQ ID NO:8 or SEQ ID NO:9 and/or is capable of hybridizing to one or more of SEQ ID NO:1, SEQ ID NO:2, SEQ ID NO:3, SEQ ID NO:4, SEQ ID NO:5, SEQ ID NO:6, SEQ ID NO:7, SEQ ID NO:8 or SEQ ID NO:9 or their complementary forms under low stringency conditions at 42° C. and wherein said nucleic acid molecule is differentially expressed in one or more of stomach, liver or hypothalamus tissue under fed or unfed or diabetic or non-diabetic conditions.

Higher similarities are also contemplated by the present invention such as greater than about 40%, 50%, 60%, 70%, 80%, 90%, 95%, 96%, 97%, 98%, 99% or above.

An expression product includes an RNA molecule such as an mRNA transcript as well as a protein. Some genes are non-protein encoding genes and produce mRNA or other RNA molecules and are involved in regulation by RNA:DNA, RNA:RNA or RNA:protein interaction. The RNA (e.g. mRNA) may act directly or via the induction of other molecules such as RNAi or via products mediated from splicing events (e.g. exons or introns). Other genes encode mRNA transcripts which are then translated into proteins. A protein includes a polypeptide. The differentially expressed nucleic acid molecules, therefore, may encode mRNAs only or, in addition, proteins. Both mRNAs and proteins are forms of “expression products”.

Reference herein to similarity is generally at a level of comparison of at least 15 consecutive or substantially consecutive nucleotides. It is particularly convenient, however, to determine similarity by comparing a total or complete sequence, after optimal alignment.

The term “similarity” as used herein includes exact identity between compared sequences at the nucleotide level. Where there is non-identity at the nucleotide level, “similarity” includes differences between sequences which may encode different amino acids that are nevertheless related to each other at the structural, functional, biochemical and/or conformational levels. In a particularly preferred embodiment, nucleotide sequence comparisons are made at the level of identity rather than similarity.

Terms used to describe sequence relationships between two or more polynucleotides include “reference sequence”, “comparison window”, “sequence similarity”, “sequence identity”, “percentage of sequence similarity”, “percentage of sequence identity”, “substantially similar” and “substantial identity”. A “reference sequence” is at least 12 but frequently 15 to 18 and often at least 25 or above, such as 30 monomer units in length. Because two polynucleotides may each comprise (1) a sequence (i.e. only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of typically 12 contiguous residues that is compared to a reference sequence. The comparison window may comprise additions or deletions (i.e. gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e. resulting in the highest percentage homology over the comparison window) generated by any of the various methods selected. Reference also may be made to the BLAST family of programs as for example disclosed by Altschul et al. (Nucl. Acids Res. 25: 3389, 1997). A detailed discussion of sequence analysis can be found in Unit 19.3 of Ausubel et al. (“Current Protocols in Molecular Biology” John Wiley & Sons Inc, Chapter 15, 1994-1998). A range of other algorithms may be used to compare the nucleotide and amino acid sequences such as but not limited to PILEUP, CLUSTALW, SEQUENCHER or VectorNTI.

The terms “sequence similarity” and “sequence identity” as used herein refer to the extent that sequences are identical or functionally or structurally similar on a nucleotide-by-nucleotide basis over a window of comparison. Thus, a “percentage of sequence identity”, for example, is calculated by comparing two optimally aligned sequences over the window of comparison, determining the number of positions at which the identical nucleic acid base (e.g. A, T, C, G, I) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” will be understood to mean the “match percentage” calculated by the DNASIS computer program (Version 2.5 for windows; available from Hitachi Software engineering Co., Ltd., South San Francisco, Calif., USA) using standard defaults as used in the reference manual accompanying the software. Similar comments apply in relation to sequence similarity.

Reference herein to a low stringency includes and encompasses from at least about 0 to at least about 15% v/v formamide and from at least about 1 M to at least about 2 M salt for hybridization, and at least about 1 M to at least about 2 M salt for washing conditions. Generally, low stringency is at least from about 25-30° C. to about 42° C. The temperature may be altered and higher temperatures used to replace formamide and/or to give alternative stringency conditions. Alternative stringency conditions may be applied where necessary, such as medium stringency, which includes and encompasses from at least about 16% v/v to at least about 30% v/v formamide and from at least about 0.5 M to at least about 0.9 M salt for hybridization, and at least about 0.5 M to at least about 0.9 M salt for washing conditions, or high stringency, which includes and encompasses from at least about 31% v/v to at least about 50% v/v formamide and from at least about 0.01 M to at least about 0.15 M salt for hybridization, and at least about 0.01 M to at least about 0.15 M salt for washing conditions. In general, washing is carried out at a temperature below the melting point, Tₘ, calculated as Tₘ=69.3+0.41 (G+C)%, where G=and C=(Marmur and Doty, J. Mol. Biol. 5: 109, 1962). However, the Tₘ of a duplex DNA decreases by 1° C. with every increase of 1% in the number of mismatch base pairs (Bonner and Laskey, Eur. J. Biochem. 46: 83, 1974). Formamide is optional in these hybridization conditions. In a particular embodiment, levels of stringency are defined as follows: low stringency is 6×SSC buffer, 0.1% w/v SDS at 25-42° C.; a moderate stringency is 2×SSC buffer, 0.1% w/v SDS at a temperature in the range 20° C. to 65° C.; high stringency is 0.1×SSC buffer, 0.1% w/v SDS at a temperature of at least 65° C.

The nucleotide sequence or amino acid sequence of the present invention may correspond to exactly the same sequence of the naturally occurring gene (or corresponding cDNA) or protein or other expression product or may carry one or more nucleotide or amino acid substitutions, additions and/or deletions. The nucleotide sequences set forth in SEQ ID NO:1 (AGT-119), SEQ ID NO:2 (AGT-120), SEQ ID NO:3 (AGT-121), SEQ ID NO:5 (AGT-122), SEQ ID NO:6 (AGT-422), SEQ ID NO:7 (AGT-123), SEQ ID NO:8 (AGT-504 genomic) or SEQ ID NO:9 (AGT-504 cDNA) correspond to novel genes referred to in parenthesis. The corresponding expression products are AGT-119, AGT-120, AGT-121, AGT-122, AGT-422, AGT-123 and AGT-504. Reference herein to AGT-19, AGT-120, AGT-121, AGT-122, AGT-422, AGT-123 and AGT-504 includes, where appropriate, reference to the genomic gene or cDNA as well as any naturally occurring or induced derivatives. For example, a genomic form of AGT-504 is represented as SEQ ID NO:8. The present invention further encompasses mutants, fragments, parts and portions of the nucleotide sequence corresponding to AGT-119, AGT-120, AGT-121, AGT-122, AGT-422, AGT-123 and AGT-504.

Another aspect of the present invention provides a nucleic acid molecule or derivative, homolog or analog thereof comprising a nucleotide sequence encoding, or a nucleotide sequence complementary to a sequence encoding an expression product wherein said nucleotide sequence is substantially as set forth in SEQ ID NO:1 (AGT-119) or a derivative, homolog or mimetic thereof or having at least about 30% similarity to all or part of SEQ ID NO:1 or a nucleotide sequence capable of hybridizing to SEQ ID NO:1 or its complementary form under low stringency conditions.

Yet another aspect of the present invention provides a nucleic acid molecule or derivative, homolog or analog thereof comprising a nucleotide sequence encoding, or a nucleotide sequence complementary to a sequence encoding an expression product wherein said nucleotide sequence is substantially as set forth in SEQ ID NO:2 (AGT-120) or a derivative, homolog or mimetic thereof or having at least about 30% similarity to all or part of SEQ ID NO:2 or a nucleotide sequence capable of hybridizing to SEQ ID NO:2 or its complementary form under low stringency conditions.

Still yet another aspect of the present invention provides a nucleic acid molecule or derivative, homolog or analog thereof comprising a nucleotide sequence encoding, or a nucleotide sequence complementary to a sequence encoding an expression product wherein said nucleotide sequence is substantially as set forth in SEQ ID NO:3 (AGT-121) or a derivative, homolog or mimetic thereof or having at least about 30% similarity to all or part of SEQ ID NO:3 or a nucleotide sequence capable of hybridizing to SEQ ID NO:3 or their complementary forms under low stringency conditions.

Even yet another aspect of the present invention provides a nucleic acid molecule or derivative, homolog or analog thereof comprising a nucleotide sequence encoding, or a nucleotide sequence complementary to a sequence encoding an expression product wherein said nucleotide sequence is substantially as set forth in SEQ ID NO:5 (AGT-122) or a derivative, homolog or mimetic thereof or having at least about 30% similarity to all or part of SEQ ID NO:5 or a nucleotide sequence capable of hybridizing to SEQ ID NO:5 or its complementary form under low stringency conditions.

Even still another aspect of the present invention provides a nucleic acid molecule or derivative, homolog or analog thereof comprising a nucleotide sequence encoding, or a nucleotide sequence complementary to a sequence encoding an expression product wherein said nucleotide sequence is substantially as set forth in SEQ ID NO:6 (AGT-422) or a derivative, homolog or mimetic thereof or having at least about 30% similarity to all or part of SEQ ID NO:6 or a nucleotide sequence capable of hybridizing to SEQ ID NO:6 or its complementary form under low stringency conditions.

Another aspect of the present invention provides a nucleic acid molecule or derivative, homolog or analog thereof comprising a nucleotide sequence encoding, or a nucleotide sequence complementary to a sequence encoding an expression product wherein said nucleotide sequence is substantially as set forth in SEQ ID NO:7 (AGT-123) or a derivative, homolog or mimetic thereof or having at least about 30% similarity to all or part of SEQ ID NO:7 or a nucleotide sequence capable of hybridizing to SEQ ID NO:7 or its complementary form under low stringency conditions.

A further aspect of the present invention provides a nucleic acid molecule or derivative, homolog or analog thereof comprising a nucleotide sequence encoding, or a nucleotide sequence complementary to a sequence encoding an expression product wherein said nucleotide sequence is substantially as set forth in SEQ ID NO:8 (AGT-504 genomic) or a derivative, homolog or mimetic thereof or having at least about 30% similarity to all or part of SEQ ID NO:8 or a nucleotide sequence capable of hybridizing to SEQ ID NO:8 or its complementary form under low stringency conditions.

Yet another aspect of the present invention provides a nucleic acid molecule or derivative, homolog or analog thereof comprising a nucleotide sequence encoding, or a nucleotide sequence complementary to a sequence encoding an expression product wherein said nucleotide sequence is substantially as set forth in SEQ ID NO:9 (AGT-504 cDNA) or a derivative, homolog or mimetic thereof or having at least about 30% similarity to all or part of SEQ ID NO:9 or a nucleotide sequence capable of hybridizing to SEQ ID NO:9 or its complementary form under low stringency conditions.

The expression pattern of AGT-119, AGT-120, AGT-121, AGT-122, AGT-422, AGT-123 and AGT-504 has been determined, inter alia, to indicate an involvement in the regulation of one or more of obesity, anorexia, weight maintenance, diabetes and/or energy metabolism. In addition to the differential expression of AGT-119, AGT-120, AGT-121, AGT-122, AGT-422, AGT-123 and AGT-504 in one or more of stomach, liver or hypothalamus tissue of fed versus fasted or diabetic versus non-diabetic animals, these genes may also be expressed in other tissues including but in no way limited to brain, muscle, adipose tissue, pancreas and gastrointestinal trait. The nucleic acid molecule encoding each of AGT-119, AGT-120, AGT-121, AGT-122, AGT-422, AGT-123 or AGT-504 may be a DNA such as a cDNA sequence or a genomic DNA. A genomic sequence may also comprise exons and introns. A genomic sequence may also include a promoter region or other regulatory regions.

A homolog is considered to be a gene from another animal species which has the same or greater than 30% similarity to one of AGT-119, AGT-120, AGT-121, AGT-122, AGT-422, AGT-123 and AGT-504 and/or which has a similar function. The above-mentioned genes are exemplified herein as related to Psammomys obesus stomach, liver or hypothalamus. The present invention extends, however, to the homologous gene, as determined by nucleotide sequence and/or function, from humans, primates, livestock animals (e.g. cows, sheep, pigs, horses, donkeys), laboratory test animals (e.g. mice, guinea pigs, hamsters, rabbits), companion animals (e.g. cats, dogs) and captured wild animals (e.g. rodents, foxes, deer, kangaroos).

The nucleic acids of the present invention and in particular AGT-119, AGT-120, AGT-121, AGT-122, AGT-422, AGT-123 and AGT-504 and their derivatives and homologs may be in isolated or purified form and/or may be ligated to a vector such as an expression vector. Expression may be in a eukaryotic cell line (e.g. mammalian, insect or yeast cells) or in prokaryote cells (e.g. E. coli) or in both. “Isolated” refers to a nucleic acid molecule having undergone at least one purification step and this is conveniently defined, for example, by a composition comprising at least about 10% subject nucleic acid molecule, at least about 20%, at least about 30%, at least about 40-50%, at least about 60-70%, at least about 80-90% or greater of subject nucleic acid molecule relative to other components as determined by molecular weight, encoding activity, nucleotide sequence, base composition or other convenient means. The nucleic acid molecule of the present invention may also be considered, in a particular embodiment, to be biologically pure. The nucleic acid molecule may be ligated to an expression vector capable of expression in a prokaryotic cell (e.g. E. coli) or a eukaryotic cell (e.g. yeast cells, fungal cells, insect cells, mammalian cells or plant cells). The nucleic acid molecule may be ligated or fused or otherwise associated with a nucleic acid molecule encoding another entity such as, for example, a signal peptide. It may also comprise additional nucleotide sequence information fused, linked or otherwise associated with it either at the 3′ or 5′ terminal portions or at both the 3′ and 5′ terminal portions. The nucleic acid molecule may also be part of a vector, such as an expression vector.

The derivatives of the nucleic acid molecule of the present invention include oligonucleotides, PCR primers, antisense molecules, molecules suitable for use in co-suppression and fusion nucleic acid molecules. Ribozymes and DNAzymes are also contemplated by the present invention directed to AGT-119, AGT-120, AGT-121, AGT-122, AGT-422, AGT-123 and AGT-504 or their mRNAs. Derivatives and homologs of AGT-119, AGT-120, AGT-121, AGT-122, AGT-422, AGT-123 and AGT-504 are conveniently encompassed by those nucleotide sequences capable of hybridizing to one or more of SEQ ID NO:1, SEQ ID NO:2, SEQ ID NO:3, SEQ ID NO:5, SEQ ID NO:6, SEQ ID NO:7, SEQ ID NO:8 or SEQ ID NO:9 or their complementary forms under low stringency conditions.

Derivatives include fragments, parts, portions, mutants, variants and mimetics from natural, synthetic or recombinant sources including fusion nucleic acid molecules. Derivatives may be derived from insertion, deletion or substitution of nucleotides.

Another aspect of the present invention provides an isolated expression product or a derivative, homolog, analog or mimetic thereof which is produced in larger or lesser amounts in one or more of stomach, liver or hypothalamus tissue in obese animals compared to lean animals or in fed (including re-fed) compared to fasted animals or in animals under diabetic compared to non-diabetic conditions.

An expression product, as indicated above, may be RNA or protein. Insofar as the product is a protein, derivatives include amino acid insertional derivatives such as amino and/or carboxylic terminal fusions as well as intra-sequence insertions of single or multiple amino acids. Insertional amino acid sequence variants are those in which one or more amino acid residues are introduced into a predetermined site in a protein although random insertion is also possible with suitable screening of the resulting product. Deletional variants are characterized by the removal of one or more amino acids from the sequence. Substitutional amino acid variants are those in which at least one residue in the sequence has been removed and a different residue inserted in its place. An example of substitutional amino acid variants are conservative amino acid substitutions. Conservative amino acid substitutions typically include substitutions within the following groups: glycine and alanine; valine, isoleucine and leucine; aspartic acid and glutamic acid; asparagine and glutamine; serine and threonine; lysine and arginine; and phenylalanine and tyrosine. Additions to amino acid sequences include fusions with other peptides, polypeptides or proteins.

Chemical and functional equivalents of protein forms of the expression products AGT-119, AGT-120, AGT-121, AGT-122, AGT-422, AGT-123 or AGT-504 should be understood as molecules exhibiting any one or more of the functional activities of these molecules and may be derived from any source such as being chemically synthesized or identified via screening processes such as natural product screening or screening of chemical libraries.

The derivatives include fragments having particular epitopes or parts of the entire protein fused to peptides, polypeptides or other proteinaceous or non-proteinaceous molecules.

Reference herein to AGT-119, AGT-120, AGT-121, AGT-122, AGT-422, AGT-123 or AGT-504 includes reference to isolated or purified naturally occurring AGT-119, AGT-120, AGT-121, AGT-122, AGT-422, AGT-123 or AGT-504 as well as any derivatives, homologs, analogs and mimetics thereof. Derivatives include parts, fragments and portions of AGT-119, AGT-120, AGT-121, AGT-122, AGT-422, AGT-123 and AGT-504 as well as single and multiple amino acid substitutions, deletions and/or additions to AGT-119, AGT-120, AGT-121, AGT-122, AGT-422, AGT-123 and AGT-504 when the expression products are proteins. A derivative of AGT-119, AGT-120, AGT-121, AGT-122, AGT-422, AGT-123 or AGT-504 is conveniently encompassed by molecules encoded by a nucleotide sequence capable of hybridizing to SEQ ID NO:1, SEQ ID NO:2, SEQ ID NO:3, SEQ ID NO:5, SEQ ID NO:6, SEQ ID NO:7, SEQ ID NO:8 or SEQ ID NO:9 under low stringency conditions.

Other derivatives of AGT-119, AGT-120, AGT-121, AGT-122, AGT-422, AGT-123 and AGT-504 include chemical analogs. Analogs of AGT-119, AGT-120, AGT-121, AGT-122, AGT-422, AGT-123 and AGT-504 contemplated herein include, but are not limited to, modifications to side chains, incorporation of unnatural amino acids and/or their derivatives during peptide, polypeptide or protein synthesis and the use of crosslinkers and other methods which impose confirmational constraints on the proteinaceous molecule or their analogs.

Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2, 4, 6-trinitrobenzene sulfonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivitization, for example, to a corresponding amide.

Sulphydryl groups may be modified by methods such as carboxymethylation with iodoacetic acid or iodoacetamide; performic acid oxidation to cysteic acid; formation of mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carbethoxylation with diethylpyrocarbonate.

Examples of incorporating unnatural amino acids and derivatives during peptide synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. A list of unnatural amino acid, contemplated herein is shown in Table 3.

TABLE 3
Codes for non-conventional amino acids

| Non-conventional | | Non-conventional | |
|---|---|---|---|
| amino acid | Code | amino acid | Code |
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgln |
| carboxylate | | L-N-methylglutamic acid | Nmglu |
| cyclohexylalanine | Chexa | L-Nmethylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcylcopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cylcododecylglycine | Ncdod |
| D-N-methylalanine | Dnmala | N-cyclooctylglycine | Ncoct |
| D-N-methylarginine | Dnmarg | N-cyclopropylglycine | Ncpro |
| D-N-methylasparagine | Dnmasn | N-cycloundecylglycine | Ncund |
| D-N-methylaspartate | Dnmasp | N-(2,2-diphenylethyl)glycine | Nbhm |
| D-N-methylcysteine | Dnmcys | N-(3,3-diphenylpropyl)glycine | Nbhe |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl))glycine | Nser |
| D-N-methylisoleucine | Dnmile | N-(imidazolylethyl))glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | Mser | L-α-methylthreonine | Mthr |
| L-α-methyltryptophan | Mtrp | L-α-methyltyrosine | Mtyr |
| L-α-methylvaline | Mval | L-N-methylhomophenylalanine | Nmhphe |
| N-(N-(2,2-diphenylethyl) | Nnbhm | N-(N-(3,3-diphenylpropyl) | Nnbhe |
| carbamylmethyl)glycine | | carbamylmethyl)glycine | |
| 1-carboxy-1-(2,2-diphenyl- | Nmbc | | |
| ethylamino)cyclopropane | | | |

Crosslinkers can be used, for example, to stabilize 3D conformations, using homo-bifunctional crosslinkers such as the bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety such as maleimido or dithio moiety (SH) or carbodiimide (COOH). In addition, peptides can be conformationally constrained by, for example, incorporation of C_{α} and N_{β}-methylamino acids, introduction of double bonds between C_{α} and C_{β} atoms of amino acids and the formation of cyclic peptides or analogs by introducing covalent bonds such as forming an amide bond between the N and C termini, between two side chains or between a side chain and the N or C terminus.

All such modifications may also be useful in stabilizing the AGT-119, AGT-120, AGT-121, AGT-122, AGT-422, AGT-123 and AGT-504 molecule for use in in vivo administration protocols or for diagnostic purposes.

As stated above, the expression product may be a RNA or protein.

The term “protein” should be understood to encompass peptides, polypeptides and proteins. The protein may be glycosylated or unglycosylated and/or may contain a range of other molecules fused, linked, bound or otherwise associated to the protein such as amino acids, lipids, carbohydrates or other peptides, polypeptides or proteins. Reference hereinafter to a “protein” includes a protein comprising a sequence of amino acids as well as a protein associated with other molecules such as amino acids, lipids, carbohydrates or other peptides, polypeptides or proteins.

In a particular embodiment, the expression product is encoded by a sequence of nucleotides comprising SEQ ID NO:1 or a derivative, homolog or analog thereof including a nucleotide sequence having at least about 30% similarity to SEQ ID NO:1 or a nucleotide sequence capable of hybridizing to SEQ ID NO:1 or its complementary form under low stringency conditions.

In another particular embodiment, the expression product is encoded by a sequence of nucleotides comprising SEQ ID NO:2 or a derivative, homolog or analog thereof including a nucleotide sequence having at least about 30% similarity to SEQ ID NO:2 or a nucleotide sequence capable of hybridizing to SEQ ID NO:2 or its complementary form under low stringency conditions.

In still another particular embodiment, the expression product is encoded by a sequence of nucleotides comprising SEQ ID NO:3 or a derivative homolog or analog thereof including a nucleotide sequence having at least about 30% similarity to SEQ ID NO:3 or a nucleotide sequence capable of hybridizing to SEQ ID NO:3 or their complementary form under low stringency conditions.

In yet another particular embodiment, the expression product is encoded by a sequence of nucleotides comprising SEQ ID NO:5 or a derivative homolog or analog thereof including a nucleotide sequence having at least about 30% similarity to SEQ ID NO:5 or a nucleotide sequence capable of hybridizing to SEQ ID NO:5 or its complementary form under low stringency conditions.

In another particular embodiment, the expression product is encoded by a sequence of nucleotides comprising SEQ ID NO:6 or a derivative homolog or analog thereof including a nucleotide sequence having at least about 30% similarity to SEQ ID NO:6 or a nucleotide sequence capable of hybridizing to SEQ ID NO:6 or its complementary form under low stringency conditions.

In a further particular embodiment, the expression product is encoded by a sequence of nucleotides comprising SEQ ID NO:7 or a derivative homolog or analog thereof including a nucleotide sequence having at least about 30% similarity to SEQ ID NO:7 or a nucleotide sequence capable of hybridizing to SEQ ID NO:7 or its complementary form under low stringency conditions.

In still another particular embodiment, the expression product is encoded by a sequence of nucleotides comprising SEQ ID NO:8 or a derivative homolog or analog thereof including a nucleotide sequence having at least about 30% similarity to SEQ ID NO:8 or a nucleotide sequence capable of hybridizing to SEQ ID NO:8 or its complementary form under low stringency conditions.

In yet another particular embodiment, the expression product is encoded by a sequence of nucleotides comprising SEQ ID NO:9 or a derivative homolog or analog thereof including a nucleotide sequence having at least about 30% similarity to SEQ ID NO:9 or a nucleotide sequence capable of hybridizing to SEQ ID NO:9 or its complementary form under low stringency conditions.

Higher similarities are also contemplated by the present invention such as greater than 40%, 50%, 60%, 70%, 80%, 90%, 95%, 96%, 97%, 98% or 99% or above.

Another aspect of the present invention is directed to an isolated expression product selected from the group consisting of:
(i) an mRNA or protein encoded by a novel nucleic acid molecule which molecule is differentially expressed in one or more of stomach, liver or hypothalamus tissue from Psammomys obesus animals under fed or fasting conditions or animals which are diabetic or non-diabetic or a derivative, homolog, analog, chemical equivalent or mimetic thereof.
(ii) AGT-119, AGT-120, AGT-121, AGT-122, AGT-422, AGT-123 or AGT-504 or a derivative, homolog, analog, chemical equivalent or mimetic thereof;
(iii) a protein encoded by a nucleotide sequence comprising SEQ ID NO:1 or a derivative, homolog or analog thereof or a sequence encoding an amino acid sequence having at least about 30% similarity to this sequence or a derivative, homolog, analog, chemical equivalent or mimetic of said protein;
(iv) a protein encoded by a nucleotide sequence substantially comprising SEQ ID NO:2 or a derivative, homolog or analog thereof or a sequence encoding an amino acid sequence having at least about 30% similarity to this sequence or a derivative, homolog, analog, chemical equivalent or mimetic of said protein;
(v) a protein encoded by a nucleotide sequence substantially comprising SEQ ID NO:3 or a derivative, homolog or analog thereof or a sequence encoding an amino acid sequence having at least about 30% similarity to these sequences or a derivative, homolog, analog, chemical equivalent or mimetic of said protein;
(vi) a protein comprising an amino acid sequence substantially as set forth in SEQ ID NO:4 or a derivative, homolog or analog thereof or a sequence encoding an amino acid sequence having at least about 30% similarity to these sequences or a derivative, homolog, analog, chemical equivalent or mimetic of said protein;
(vii) a protein encoded by a nucleotide sequence substantially comprising SEQ ID NO:5 or a derivative, homolog or analog thereof or a sequence encoding an amino acid sequence having at least about 30% similarity to this sequence or a derivative, homolog, analog, chemical equivalent or mimetic of said protein;
(viii) a protein encoded by a nucleotide sequence substantially comprising SEQ ID NO:6 or a derivative, homolog or analog thereof or a sequence encoding an amino acid sequence having at least about 30% similarity to this sequence or a derivative, homolog, analog, chemical equivalent or mimetic of said protein;
(ix) a protein encoded by a nucleotide sequence substantially comprising SEQ ID NO:7 or a derivative, homolog or analog thereof or a sequence encoding an amino acid sequence having at least about 30% similarity to this sequence or a derivative, homolog, analog, chemical equivalent or mimetic of said protein;
(x) a protein encoded by a nucleotide sequence substantially comprising SEQ ID NO:8 or a derivative, homolog or analog thereof or a sequence encoding an amino acid sequence having at least about 30% similarity to this sequence or a derivative, homolog, analog, chemical equivalent or mimetic of said protein;
(xi) a protein encoded by a nucleotide sequence substantially comprising SEQ ID NO:9 or a derivative, homolog or analog thereof or a sequence encoding an amino acid sequence having at least about 30% similarity to this sequence or a derivative, homolog, analog, chemical equivalent or mimetic of said protein;
(xii) a protein encoded by a nucleic acid molecule capable of hybridizing to a nucleotide sequence comprising SEQ ID NO:1 or its complementary form or a derivative, homolog or analog thereof under low stringency conditions;
(xiii) a protein encoded by a nucleic acid molecule capable of hybridizing to a nucleotide sequence comprising SEQ ID NO:2 or its complementary form or a derivative, homolog or analog thereof under low stringency conditions;
(xiv) a protein encoded by a nucleic acid molecule capable of hybridizing to a nucleotide sequence comprising SEQ ID NO:3 or their complementary forms or a derivative, homolog or analog thereof under low stringency conditions;
(xv) a protein encoded by a nucleic acid molecule capable of hybridizing to a nucleotide sequence comprising SEQ ID NO:5 or its complementary form or a derivative, homolog or analog thereof under low stringency conditions;
(xvi) a protein encoded by a nucleic acid molecule capable of hybridizing to a nucleotide sequence comprising SEQ ID NO:6 or its complementary form or a derivative, homolog or analog thereof under low stringency conditions;
(xvii) a protein encoded by a nucleic acid molecule capable of hybridizing to a nucleotide sequence comprising SEQ ID NO:7 or its complementary form or a derivative, homolog or analog thereof under low stringency conditions;
(xviii) a protein encoded by a nucleic acid molecule capable of hybridizing to a nucleotide sequence comprising SEQ ID NO:8 or its complementary form or a derivative, homolog or analog thereof under low stringency conditions; and
(xix) a protein encoded by a nucleic acid molecule capable of hybridizing to a nucleotide sequence comprising SEQ ID NO:9 or its complementary form or a derivative, homolog or analog thereof under low stringency conditions.

An example of an expression product is the amino acid sequence set forth in SEQ ID NO:4 (AGT-121).

The protein of the present invention may be in isolated form. “Isolated” refers to a protein having undergone at least one purification step and this is conveniently defined, for example, by a composition comprising at least about 10% subject protein, at least about 20%, at least about 30%, at least about 40-50%, at least about 60-70%, 80-90% or greater of subject protein relative to other components as determined by molecular weight, amino acid sequence or other convenient means. The protein of the present invention may also be considered, in a particular embodiment, to be biologically pure.

Without limiting the theory or mode of action of the present invention, the expression of AGT-119, AGT-120, AGT-121, AGT-122, AGT-422, AGT-123 and/or AGT-504 is thought to relate to regulation of body weight and glucose homeostasis. Modulation of expression of these genes is thought inter alia to regulate energy balance via effects on energy intake and also effects on carbohydrate/fat metabolism. The energy intake effects are likely to be mediated via the central nervous system but peripheral effects on the metabolism of both carbohydrate and fat are possible. The expression of these genes may also be regulated by fasting and feeding. Accordingly, regulating the expression and/or activity of these genes or their expression products provides a mechanism for regulating both body weight and energy metabolism, including carbohydrate and fat metabolism.

The identification of AGT-119, AGT-120, AGT-121, AGT-122, AGT-422, AGT-123 and AGT-504 permits the generation of a range of therapeutic molecules capable of modulating expression of AGT-119, AGT-120, AGT-121, AGT-122, AGT-422, AGT-123 and AGT-504 or modulating the activity of AGT-119, AGT-120, AGT-121, AGT-122, AGT-422, AGT-123 and AGT-504. Modulators contemplated by the present invention include agonists and antagonists of AGT-119, AGT-120, AGT-121, AGT-122, AGT-422, AGT-123 and AGT-504 expression. Antagonists of AGT-119, AGT-120, AGT-121, AGT-122, AGT-422, AGT-123 and AGT-504 expression include antisense molecules, ribozymes and co-suppression molecules (including any molecules which induce RNAi). Agonists include molecules which increase promoter activity or which interfere with negative regulatory mechanisms. Antagonists of AGT-119, AGT-120, AGT-121, AGT-122, AGT-422, AGT-123 and AGT-504 include antibodies and inhibitor peptide fragments. Such molecules may first need to be modified to enable such molecules to penetrate cell membranes. Alternatively, viral agents may be employed to introduce genetic elements to modulate expression of AGT-119, AGT-120, AGT-121, AGT-122, AGT-422, AGT-123 and AGT-504. In so far as AGT-119, AGT-120, AGT-121, AGT-122, AGT-422, AGT-123 and AGT-504 act in association with other genes such as the ob gene which encodes leptin, the therapeutic molecules may target AGT-119, AGT-120, AGT-121, AGT-122, AGT-422, AGT-123 and AGT-504 and ob genes or their translation products.

The present invention contemplates, therefore, a method for modulating expression of AGT-119, AGT-120, AGT-121, AGT-122, AGT-422, AGT-123 and AGT-504 in a mammal, said method comprising contacting the AGT-119, AGT-120, AGT-121, AGT-122, AGT-422, AGT-123 and AGT-504 gene with an effective amount of a modulator of AGT-119, AGT-120, AGT-121, AGT-122, AGT-422, AGT-123 and AGT-504 expression for a time and under conditions sufficient to up-regulate or down-regulate or otherwise modulate expression of AGT-119, AGT-120, AGT-121, AGT-122, AGT-422, AGT-123 and AGT-504.

For example, a nucleic acid molecule encoding AGT-119, AGT-120, AGT-121, AGT-122, AGT-422, AGT-123 and AGT-504 or a derivative or homolog thereof may be introduced into a cell to enhance the ability of that cell to produce AGT-119, AGT-120, AGT-121, AGT-122, AGT-422, AGT-123 and AGT-504, conversely, AGT-119, AGT-120, AGT-121, AGT-122, AGT-422, AGT-123 and AGT-504 sense and/or antisense sequences such as oligonucleotides may be introduced to decrease expression of the genes at the level of transcription, post-transcription or translation. Sense sequences preferably encode hair pin RNA molecules or double-stranded RNA molecules.

Another aspect of the present invention contemplates a method of modulating activity of AGT-119, AGT-120, AGT-121, AGT-122, AGT-422, AGT-123 and AGT-504 in a mammal, said method comprising administering to said mammal a modulating effective amount of a molecule for a time and under conditions sufficient to increase or decrease AGT-119, AGT-120, AGT-121, AGT-122, AGT-422, AGT-123 and AGT-504 activity. The molecule may be a proteinaceous molecule or a chemical entity and may also be a derivative of AGT-119, AGT-120, AGT-121, AGT-122, AGT-422, AGT-123 and AGT-504 or its ligand.

Modulating levels of AGT-119, AGT-120, AGT-121, AGT-122, AGT-422, AGT-123 and AGT-504 expression or AGT-119, AGT-120, AGT-121, AGT-122, AGT-422, AGT-123 and/or AGT-504 activity or function is important in the treatment of a range of conditions such as obesity, anorexia, energy imbalance, diabetes, metabolic syndrome, dyslipidemia, hypertension and insulin resistance. It may also be useful in the agricultural industry to assist in the generation of leaner animals, or where required, more obese animals. Accordingly, mammals contemplated by the present invention include but are not limited to humans, primates, livestock animals (e.g. pigs, sheep, cows, horses, donkeys), laboratory test animals (e.g. mice, rats, guinea pigs, hamsters, rabbits), companion animals (e.g. dogs, cats) and captured wild animals (e.g. foxes, kangaroos, deer). A particular host is a human, primate or livestock animal.

Accordingly, the present invention contemplates therapeutic and prophylactic use of AGT-119, AGT-120, AGT-121, AGT-122, AGT-422, AGT-123 and/or AGT-504 expression products or AGT-119, AGT-120, AGT-121, AGT-122, AGT-422, AGT-123 and AGT-504 genetic mutants and/or agonists or antagonists agents thereof.

The present invention contemplates, therefore, a method of modulating expression of AGT-119, AGT-120 AGT-121, AGT-122, AGT-422, AGT-123 and/or AGT-504 in a mammal, said method comprising contacting the AGT-119, AGT-120, AGT-121, AGT-122, AGT-422, AGT-123 and/or AGT-504 genes with an effective amount of an agent for a time and under conditions sufficient to up-regulate, down-regulate or otherwise module expression of AGT-119, AGT-120, AGT-121, AGT-122, AGT-422, AGT-123 and AGT-504.

Another aspect of the present invention contemplates a method of modulating activity of AGT-119, AGT-120, AGT-121, AGT-122, AGT-422, AGT-123 and/or AGT-504 in a subject, said method comprising administering to said subject a modulating effective amount of an agent for a time and under conditions sufficient to increase or decrease AGT-119, AGT-120, AGT-121, AGT-122, AGT-422, AGT-123 and/or AGT-504 activity or function.

Modulation of activity by the administration of an agent to a mammal can be achieved by one of several techniques, including, but in no way limited to, introducing into a mammal a proteinaceous or non-proteinaceous molecule which:
(i) modulates expression of AGT-119, AGT-120, AGT-121, AGT-122, AGT-422, AGT-123 and/or AGT-504;
(ii) functions as an antagonist of AGT-119, AGT-120, AGT-121, AGT-122, AGT-422, AGT-123 and/or AGT-504; and/or
(iii) functions as an agonist of AGT-119, AGT-120, AGT-121, AGT-122, AGT-422, AGT-123 and/or AGT-504.

The molecules which may be administered to a mammal in accordance with the present invention may also be linked to a targeting means such as a monoclonal antibody, which provides specific delivery of these molecules to the target cells.

A further aspect of the present invention relates to the use of the invention in relation to mammalian disease conditions. For example, the present invention is particularly useful in a therapeutic or prophylactic treatment of obesity, anorexia, diabetes or energy imbalance.

Accordingly, another aspect of the present invention relates to a method of treating a mammal suffering from a condition characterized by one or more symptoms of obesity, anorexia, diabetes and/or energy imbalance, said method comprising administering to said mammal an effective amount of an agent for a time and under conditions sufficient to modulate the expression of AGT-119, AGT-120, AGT-121, AGT-122, AGT-422, AGT-123 and/or AGT-504 or sufficient to modulate the activity of AGT-119, AGT-120, AGT-121, AGT-122, AGT-422, AGT-123 and/or AGT-504.

In another aspect, the present invention relates to a method of treating a mammal suffering from a disease condition characterized by one or more symptoms of obesity, anorexia, diabetes or energy imbalance, said method comprising administering to said mammal an effective amount of AGT-119, AGT-120, AGT-121, AGT-122, AGT-422, AGT-123 and/or AGT-504 or AGT-119, AGT-120, AGT-121, AGT-122, AGT-422, AGT-123 and/or AGT-504.

An agent includes proteinaceous or non-proteinaceous molecules such as antibodies, natural products, chemical entities or nucleic acid molecules (including antisense molecules, sense molecules, ribozymes, ds-RNA molecules or DNA-targeting molecules).

An “effective amount” means an amount necessary to at least partly attain the desired immune response (e.g. against AGT-119, AGT-120, AGT-121, AGT-122, AGT-422, AGT-123 or AGT-504) or to delay the onset or inhibit progression or halt altogether the onset or progression of a particular condition.

In accordance with these methods, AGT-119, AGT-120, AGT-121, AGT-122, AGT-422, AGT-123 and/or AGT-504 or AGT-119, AGT-120, AGT-121, AGT-122, AGT-422, AGT-123 and/or AGT-504 or agents capable of modulating the expression or activity of said molecules may be co-administered with one or more other compounds or other molecules. By “co-administered” is meant simultaneous administration in the same formulation or in two different formulations via the same or different routes or sequential administration by the same or different routes. By “sequential” administration is meant a time difference of from seconds, minutes, hours or days between the administration of the two types of molecules. These molecules may be administered in any order.

In yet another aspect, the present invention relates to the use of an agent capable of modulating the expression of AGT-119, AGT-120, AGT-121, AGT-122, AGT-422, AGT-123 and/or AGT-504 or a derivative, homolog or analog thereof in the manufacture of a medicament for the treatment of a condition characterized by obesity, anorexia, weight maintenance, diabetes and/or energy imbalance.

In still yet another aspect, the present invention relates to the use of an agent capable of modulating the activity of AGT-119, AGT-120, AGT-121, AGT-122, AGT-422, AGT-123 and/or AGT-504 or a derivative, homolog, analog, chemical equivalent or mimetic thereof in the manufacture of a medicament for the treatment of a condition characterized by obesity, anorexia, weight maintenance, diabetes and/or energy imbalance.

A further aspect of the present invention relates to the use of AGT-119, AGT-120, AGT-121, AGT-122, AGT-422, AGT-123 and/or AGT-504 or derivative, homolog or analog thereof or AGT-119, AGT-120, AGT-121, AGT-122, AGT-422, AGT-123 and/or AGT-504 or derivative, homolog, analog, chemical equivalent or mimetic thereof in the manufacture of a medicament for the treatment of a condition characterized by obesity, anorexia, weight maintenance, diabetes and/or energy imbalance.

Still yet another aspect of the present invention relates to agents for use in modulating the expression of AGT-119, AGT-120, AGT-121, AGT-122, AGT-422, AGT-123 and/or AGT-504 or a derivative, homolog or analog thereof.

A further aspect relates to agents for use in modulating AGT-119, AGT-120, AGT-121, AGT-122, AGT-422, AGT-123 and/or AGT-504 activity or a derivative, homolog, analog, chemical equivalent or mimetic thereof.

Still another aspect of the present invention relates to AGT-119, AGT-120, AGT-121, AGT-122, AGT-422, AGT-123 and/or AGT-504 or derivative, homolog or analog thereof or AGT-119, AGT-120, AGT-121, AGT-122, AGT-422, AGT-123 and/or AGT-504 or derivative, homolog, analog, chemical equivalent or mimetic thereof for use in treating a condition characterized by one or more symptoms of obesity, anorexia, weight maintenance, diabetes and/or energy imbalance.

In a related aspect of the present invention, the mammal undergoing treatment may be a human or an animal in need of therapeutic or prophylactic treatment.

Accordingly, the present invention contemplates in one embodiment a composition comprising a modulator of AGT-119, AGT-120, AGT-121, AGT-122, AGT-422, AGT-123 and AGT-504 expression or AGT-119, AGT-120, AGT-121, AGT-122, AGT-422, AGT-123 and AGT-504 activity and one or more pharmaceutically acceptable carriers and/or diluents. In another embodiment, the composition comprises AGT-119, AGT-120, AGT-121, AGT-122, AGT-422, AGT-123 and AGT-504 or a derivative, homolog, analog or mimetic thereof and one or more pharmaceutically acceptable carriers and/or diluents. The compositions may also comprise leptin or modulations of leptin activity or ob expression.

For brevity, all such components of such a composition are referred to as “active components”.

The compositions of active components in a form suitable for injectable use include sterile aqueous solutions (where water soluble) and sterile powders for the extemporaneous preparation of sterile injectable solutions. In all cases, the form must be sterile and must be fluid to the extent that easy syringability exists. It must be stable under the conditions of manufacture and storage and must be preserved against the contaminating action of microorganisms such as bacteria and fungi.

The carrier can be a solvent or other medium containing, for example, water, ethanol, polyol (for example, glycerol, propylene glycol and liquid polyethylene glycol, and the like), suitable mixtures thereof, and vegetable oils.

The preventions of the action of microorganisms can be brought about by various antibacterial and antifungal agents, for example, parabens, chlorobutanol, phenol, sorbic acid, thirmerosal and the like. In many cases, it will be desirable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active components in the required amount in the appropriate solvent with optionally other ingredients, as required, followed by sterilization by, for example, filter sterilization, irradiation or other convenient means. In the case of sterile powders for the preparation of sterile injectable solutions, the preferred methods of preparation are vacuum drying and the freeze-drying technique which yield a powder of the active ingredient plus any additional desired ingredient from a previously sterile-filtered solution thereof.

When AGT-119, AGT-120, AGT-121, AGT-122, AGT-422, AGT-123 and AGT-504 or AGT-119, AGT-120, AGT-121, AGT-122, AGT-422, AGT-123 and AGT-504 are suitably protected, they may be orally administered, for example, with an inert diluent or with an assimilable edible carrier, or it may be enclosed in a hard or soft shell gelatin capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 80% of the weight of the unit. The amount of active compound in such therapeutically useful compositions is such that a suitable dosage will be obtained. Compositions or preparations according to the present invention are prepared so that an oral dosage unit form contains between about 0.1 μg and 2000 mg of active compound.

The tablets, troches, pills, capsules and the like may also contain the following: a binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such a sucrose, lactose or saccharin may be added or a flavouring agent such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound may be incorporated into sustained-release preparations and formulations.

Pharmaceutically acceptable carriers and/or diluents include any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, use thereof in the therapeutic compositions is contemplated. Supplementary active ingredients can also be incorporated into the compositions.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutical carrier. The specification for the novel dosage unit forms of the invention are dictated by and directly dependent on (a) the unique characteristics of the active material and the particular therapeutic effect to be achieved, and (b) the limitations inherent in the art of compounding such an active material for the treatment of disease in living subjects having a diseased condition in which bodily health is impaired as herein disclosed in detail.

The principal active component may be compounded for convenient and effective administration in sufficient amounts with a suitable pharmaceutically acceptable carrier in dosage unit form. A unit dosage form can, for example, contain the principal active component in amounts ranging from 0.5 μg to about 2000 mg. Expressed in proportions, the active compound is generally present in from about 0.5 μg to about 2000 mg/ml of carrier. In the case of compositions containing supplementary active ingredients, the dosages are determined by reference to the usual dose and manner of administration of the said ingredients.

In general terms, effective amounts of AGT-119, AGT-120, AGT-121, AGT-122, AGT-422, AGT-123 and AGT-504 or AGT-119, AGT-120, AGT-121, AGT-122, AGT-422, AGT-123 and AGT-504 will range from 0.01 ng/kg/body weight to above 10,000 mg/kg/body weight. Alternative amounts range from 0.1 ng/kg/body weight to above 1000 mg/kg/body weight. The active ingredients may be administered per minute, hour, day, week, month or year depending on the condition being treated. The route of administration may vary and includes intravenous, intraperitoneal, sub-cutaneous, intramuscular, intranasal, via suppository, via infusion, via drip, orally or via other convenient means.

The pharmaceutical composition may also comprise genetic molecules such as a vector capable of transfecting target cells where the vector carries a nucleic acid molecule capable of modulating AGT-119, AGT-120, AGT-121, AGT-122, AGT-422, AGT-123 and AGT-504 expression or AGT-119, AGT-120, AGT-121, AGT-122, AGT-422, AGT-123 and AGT-504 activity. The vector may, for example, be a viral vector.

Still another aspect of the present invention is directed to antibodies to AGT-119, AGT-120, AGT-121, AGT-122, AGT-422, AGT-123 and AGT-504 and their derivatives and homologs insofar as AGT-119, AGT-120, AGT-121, AGT-122, AGT-422, AGT-123 and AGT-504 are proteins. Such antibodies may be monoclonal or polyclonal and may be selected from naturally occurring antibodies to AGT-119, AGT-120. AGT-121, AGT-122, AGT-422, AGT-123 and AGT-504 or may be specifically raised to AGT-119, AGT-120, AGT-121, AGT-122, AGT-422, AGT-123 and AGT-504 or derivatives or homologs thereof. In the case of the latter, AGT-119, AGT-120, AGT-121, AGT-122, AGT-422, AGT-123 and AGT-504 or their derivatives or homologs may first need to be associated with a carrier molecule. The antibodies and/or recombinant AGT-119, AGT-120, AGT-121, AGT-122, AGT-422, AGT-123 and AGT-504 or their derivatives of the present invention are particularly useful as therapeutic or diagnostic agents. An antibody “to” a molecule includes an antibody specific for said molecule.

AGT-119, AGT-120, AGT-121, AGT-122, AGT-422, AGT-123 and AGT-504 and their derivatives can be used to screen for naturally occurring antibodies to AGT-119, AGT-120, AGT-121, AGT-122, AGT-422, AGT-123 and AGT-504 which may occur in certain autoimmune diseases. Alternatively, specific antibodies can be used to screen for AGT-119, AGT-120, AGT-121, AGT-122, AGT-422, AGT-123 and AGT-504. Techniques for such assays are well known in the art and include, for example, sandwich assays and ELISA.

Antibodies to AGT-119, AGT-120, AGT-121, AGT-122, AGT-422, AGT-123 and AGT-504 of the present invention may be monoclonal or polyclonal and may be selected from naturally occurring antibodies to the AGT-19, AGT-120, AGT-121, AGT-122, AGT-422, AGT-123 and AGT-504 or may be specifically raised to the AGT-119, AGT-120 and AGT-121 or their derivatives. In the case of the latter, the AGT-119, AGT-120, AGT-121, AGT-122, AGT-422, AGT-123 and AGT-504 protein may need first to be associated with a carrier molecule. Alternatively, fragments of antibodies may be used such as Fab fragments. Furthermore, the present invention extends to recombinant and synthetic antibodies and to antibody hybrids. A “synthetic antibody” is considered herein to include fragments and hybrids of antibodies. The antibodies of this aspect of the present invention are particularly useful for immunotherapy and may also be used as a diagnostic tool or as a means for purifying AGT-119, AGT-120, AGT-121, AGT-122, AGT-422, AGT-123 and AGT-504.

For example, specific antibodies can be used to screen for AGT-119, AGT-120, AGT-121, AGT-122, AGT-422, AGT-123 and AGT-504 proteins. The latter would be important, for example, as a means for screening for levels of AGT-119, AGT-120, AGT-121, AGT-122, AGT-422, AGT-123 and AGT-504 in a cell extract or other biological fluid or purifying AGT-119, AGT-120, AGT-121, AGT-122, AGT-422, AGT-123 and AGT-504 made by recombinant means from culture supernatant fluid. Techniques for the assays contemplated herein are known in the art and include, for example, sandwich assays and ELISA.

It is within the scope of this invention to include any second antibodies (monoclonal, polyclonal or fragments of antibodies) directed to the first mentioned antibodies discussed above. Both the first and second antibodies may be used in detection assays or a first antibody may be used with a commercially available anti-immunoglobulin antibody. An antibody as contemplated herein includes any antibody specific to any region of AGT-119, AGT-120, AGT-121, AGT-122, AGT-422, AGT-123 and AGT-504.

Both polyclonal and monoclonal antibodies are obtainable by immunization with the enzyme or protein and either type is utilizable for immunoassays. The methods of obtaining both types of sera are well known in the art. Polyclonal sera are prepared by injection of a suitable laboratory animal with an effective amount of AGT-119, AGT-120, AGT-121, AGT-122, AGT-422, AGT-123 and/or AGT-504, or antigenic parts thereof, collecting serum from the animal, and isolating specific sera by any of the known immunoadsorbent techniques. Although antibodies produced by this method are utilizable in virtually any type of immunoassay, they are generally less favoured because of the potential heterogeneity of the product.

Monoclonal antibodies may be produced in large quantities as a homogeneous product. The preparation of hybridoma cell lines for monoclonal antibody production derived by fusing an immortal cell line and lymphocytes sensitized against the immunogenic preparation can be done by techniques which are well known to those who are skilled in the art.

Another aspect of the present invention contemplates a method for detecting AGT-119, AGT-120, AGT-121, AGT-122, AGT-422, AGT-123 and AGT-504 or a derivative or homolog thereof in a biological sample from a subject, said method comprising contacting said biological sample with an antibody specific for AGT-119, AGT-120, AGT-121, AGT-122, AGT-422, AGT-123 and AGT-504 or their antigenic derivatives or homologs for a time and under conditions sufficient for a complex to form, and then detecting said complex.

The presence of the complex is indicative of the presence of AGT-119, AGT-120, AGT-121, AGT-122, AGT-422, AGT-123 and AGT-504. This assay may be quantitated or semi-quantitated to determine a propensity to develop obesity or other conditions or to monitor a therapeutic regimen.

The presence of AGT-119, AGT-120, AGT-121, AGT-122, AGT-422, AGT-123 and AGT-504 may be accomplished in a number of ways such as by Western blotting and ELISA procedures. A wide range of immunoassay techniques are available as can be seen by reference to U.S. Pat. Nos. 4,016,043, 4,424,279 and 4,018,653, which are herein incorporated by reference. These, of course, include both single-site and two-site or “sandwich” assays of the non-competitive types, as well as in the traditional competitive binding assays. These assays also include direct binding of a labelled antibody to a target. Sandwich assays are among the most useful and commonly used assays. A number of variations of the sandwich assay technique exist, and all are intended to be encompassed by the present invention. Briefly, in a typical forward assay, an unlabelled antibody is immobilized on a solid substrate and the sample to be tested brought into contact with the bound molecule. After a suitable period of incubation, for a period of time sufficient to allow formation of an antibody-AGT-119, AGT-120, AGT-121, AGT-122, AGT-422, AGT-123 and/or AGT-504 complex, a second antibody specific to the AGT-119, AGT-120, AGT-121, AGT-122, AGT-422, AGT-123 and AGT-504, labelled with a reporter molecule capable of producing a detectable signal, is then added and incubated, allowing time sufficient for the formation of another complex of antibody-AGT-119, AGT-120, AGT-121, AGT-122, AGT-422, AGT-123 and AGT-504-labelled antibody. Any unreacted material is washed away, and the presence of AGT-119, AGT-120, AGT-121, AGT-122, AGT-422, AGT-123 and AGT-504 is determined by observation of a signal produced by the reporter molecule. The results may either be qualitative, by simple observation of the visible signal, or may be quantitated by comparing with a control sample containing known amounts of hapten. Variations on the forward assay include a simultaneous assay, in which both sample and labelled antibody are added simultaneously to the bound antibody. These techniques are well known to those skilled in the art, including any minor variations as will be readily apparent. In accordance with the present invention, the sample is one which might contain AGT-19, AGT-120, AGT-121, AGT-122, AGT-422, AGT-123 and AGT-504 including cell extract, tissue biopsy or possibly serum, saliva, mucosal secretions, lymph, tissue fluid and respiratory fluid. The sample is, therefore, generally a biological sample comprising biological fluid but also extends to fermentation fluid and supernatant fluid such as from a cell culture.

The solid surface is typically glass or a polymer, the most commonly used polymers being cellulose, polyacrylamide, nylon, polystyrene, polyvinyl chloride or polypropylene. The solid supports may be in the form of tubes, beads, discs or microplates, or any other surface suitable for conducting an immunoassay. The binding processes are well-known in the art and generally consist of cross-linking covalently binding or physically adsorbing, the polymer-antibody complex to the solid surface which is then washed in preparation for the test sample. An aliquot of the sample to be tested is then added to the solid phase complex and incubated for a period of time sufficient (e.g. 2-40 minutes or overnight if more convenient) and under suitable conditions (e.g. from room temperature to about 37° C.) to allow binding of any subunit present in the antibody. Following the incubation period, the antibody subunit solid phase is washed and dried and incubated with a second antibody specific for a portion of AGT-119, AGT-120, AGT-121, AGT-122, AGT-422, AGT-123 and AGT-504. The second antibody is linked to a reporter molecule which is used to indicate the binding of the second antibody to AGT-119, AGT-120, AGT-121, AGT-122, AGT-422, AGT-123 and AGT-504.

An alternative method involves immobilizing the target molecules in the biological sample and then exposing the immobilized target to specific antibody which may or may not be labelled with a reporter molecule. Depending on the amount of target and the strength of the reporter molecule signal, a bound target may be detectable by direct labelling with the antibody. Alternatively, a second labelled antibody, specific to the first antibody is exposed to the target-first antibody complex to form a target-first antibody-second antibody tertiary complex. The complex is detected by the signal emitted by the reporter molecule.

“Reporter molecule” as used in the present specification refers to a molecule which, by its chemical nature, provides an analytically identifiable signal which allows the detection of antigen-bound antibody. Detection may be either qualitative or quantitative. The most commonly used reporter molecules in this type of assay are either enzymes, fluorophores or radionuclide containing molecules (i.e. radioisotopes) and chemiluminescent molecules.

In the case of an enzyme immunoassay, an enzyme is conjugated to the second antibody, generally by means of glutaraldehyde or periodate. As will be readily recognized, however, a wide variety of different conjugation techniques exist, which are readily available to the skilled artisan. Commonly used enzymes include horseradish peroxidase, glucose oxidase, β-galactosidase and alkaline phosphatase, amongst others. The substrates to be used with the specific enzymes are generally chosen for the production, upon hydrolysis by the corresponding enzyme, of a detectable colour change. Examples of suitable enzymes include alkaline phosphatase and peroxidase. It is also possible to employ fluorogenic substrates, which yield a fluorescent product rather than the chromogenic substrates noted above. In all cases, the enzyme-labelled antibody is added to the first antibody hapten complex, allowed to bind, and then the excess reagent is washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of hapten which was present in the sample. A “reporter molecule” also extends to use of cell agglutination or inhibition of agglutination such as red blood cells on latex beads, and the like.

Alternately, fluorescent compounds, such as fluorescein and rhodamine, may be chemically coupled to antibodies without altering their binding capacity. When activated by illumination with light of a particular wavelength, the fluorochrome-labelled antibody absorbs the light energy, inducing an excited state in the molecule, followed by emission of the light at a characteristic colour visually detectable with a light microscope. The fluorescent-labelled antibody is then allowed to bind to the first antibody-hapten complex. After washing off the unbound reagent, the remaining tertiary complex is then exposed to the light of the appropriate wavelength. The fluorescence observed indicates the presence of the hapten of interest. Immunofluorescence techniques are both very well established in the art and are useful for the present method. However, other reporter molecules, such as radioisotope, chemiluminescent or bioluminescent molecules, may also be employed.

The present invention also contemplates genetic assays such as involving, for example, PCR analysis to detect AGT-119, AGT-120, AGT-121, AGT-122, AGT-422, AGT-123 and AGT-504 or their derivatives.

Real-time PCR is also useful for assaying for particular genetic molecules.

The present invention is further described by the following non-limiting Examples.

### EXAMPLE 1

### Psammomys obesus

In the following examples, Psammomys obesus rats were used for differential expression studies under different conditions. The rats were divided into three groups, based on metabolic phenotype, as follows:
Group A animals: lean
Group B animals: obese, non-diabetic
Group C animals: obese, diabetic.

### EXAMPLE 2

### Partial Sequence of Psammomys obesus AGT-119

AGT-119 was identified using differential display PCR of stomach cDNA from fed, fasted and re-fed Psammomys obesus.

The partial nucleotide sequence is as follows:

[SEQ ID NO:1]

AATGAAAGAATTGATTGATACGCAACCAAATTAGCCAGTGAGGTTAGNNN
CNGGATTATCGTGACCAGATAGGAGCCTTGGAAAATGACTAAGAAAAATG
AAAAACAGCCTAAAATGTCATTAGCCCAACAAGATGCGTTAAAACGCCTG
GATCAAGTTAGAANGCAGAAAAGCGAAAGCC.

### EXAMPLE 3

### AGT-119 Gene Expression

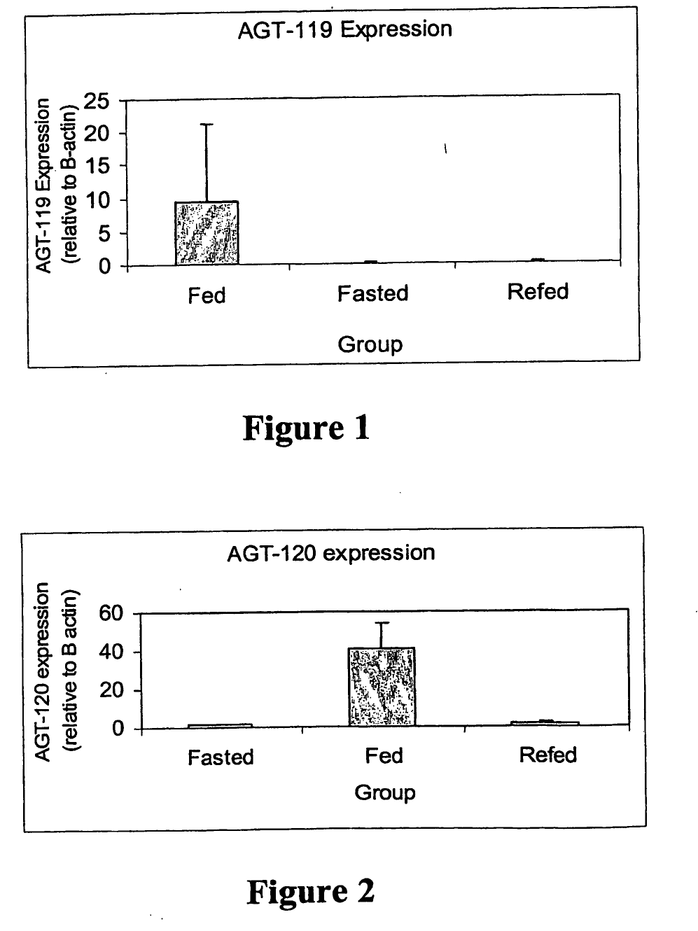

Real-time PCR analysis of AGT-119 found dramatically lower levels of expression in fasted and re-fed animals when compared to fed animals (FIG. 1). In most fasted and re-fed animals the levels of AGT-119 were undetectable. Fasting was for 16 hours, fed animals had ad libitum access to lab chow and re-fed animals were fasted for 16 hours then allowed ad libitum access to lab chow for 1 hour. These results were confirmed with two sets of primers targeting the gene of interest.

### EXAMPLE 4

### AGT-119 Sequence Homology

The AGT-119 sequence does not show significant homology with anything on the public database (BLASTN version 2.2.1).

### EXAMPLE 5

### Partial Sequence of Psammomys obesus AGT-120

AGT-120 was identified using differential display PCR of stomach cDNA from fed, fasted and re-fed Psammomys obesus.

The partial nucleotide sequence is as follows:

[SEQ ID NO:2]

GCTGATGGCGATGAATGAACACTGCGTTTGCTGGCCTTATCAATTCCCGC
TTTTTCTTGGATGAAAAAGACTAACCATGATCGGGCCCAACGGCGGAAGT
CGCTTTTGTCACCAGTAGTGATGGTGCCGGGATCAAGTGCCACGATTGAG
CGTTTCAATTCACTGATTGCGATGCTTAATAAAGATTCGCCGCACCCTCA
CAGTGTGTTAAAGATTAAAGTCATGAAAGATGGCAGTTTAAAATACAAGG
GCAGCATTAATCGCGGTGATAATGAACCCTTTATTGTGATTGGTTTTGAA
AATAATAAAGATGGCTATAGTAATATTAAGAAGCAAGCAAGCTGGCTAGA
TATTGCCTTTTATGAGATCTCGCNAACTTATAAATTTAACAACTTTAAGG
CCTTTGGCCATTCAAATGGAGGGCTGGTGTGGACATATTGGTTAGAGCAT
TATTATTCAGAGTATGAGTCAGAAATCAAAATCAAGCGGTTGATGACTTT
GGCTTCACCATTTAACTTTGACGAAGACAATCTGAATCACCGGACCCAGA
TGCTGGCTGACTTTATTAAATATCGGAAACGACTTCCAAAAACGCTCAAA
GTTTATTCACTGACTGGTGGCCAGACCTATGAATCTGACGGGATTGTTCC
TGAAAATAGTGTAGCCGCAGCCAAGTATATTTTCCAAAATCAAGTGAAGA
GCTTTATGGAAATTACGGTTACGGGTAAGCAGCTAATCACTCAGATTTAC
CGCAAAATGAACAAGTAGTGCTAGTGATGAATCCACCACTCACTAAAGAT
AATAAAAAAAAAAA.

### EXAMPLE 6

### AGT-120 Gene Expression

AGT-120 expression was significantly higher in the fed group (n=8) compared to fasted (n=12) and re-fed animals (n=8). There was no difference between fasted and re-fed animals (FIG. 2). No significant correlations were found between AGT-120 expression and stomach weight, stomach content, glucose or insulin levels.

### EXAMPLE 7

### AGT-120 Gene Homology

The AGT-120 sequence shows homology to a Lactobacillus gasseri hypothetical protein (NZ_AAAB01000011). A human homolog is yet to be identified.

### EXAMPLE 8

### Sequence of Psammomys obesus AGT-121

AGT-121 was identified using differential display PCR of hypothalamic cDNA from diabetic and non-diabetic Psammomys obesus.

AGT-121 is a hypothalamic gene that was initially identified by differential display. From primary gene expression data in fed/fasted hypothalamus, it was of interest because of the large increase in its expression in Group B and C animals, as well as the large disparity in signal between the animals.

The nucleotide sequence is as follows:

[SEQ ID NO:3]

CAGACTCCTTGGAAATTAAGGAATGCAATTCTGCCACCATGATGGAAGGA
CTGAAAAAACGTACAAGGAAGGCCTTTGGAATACGGAAGAAAGAAAAAGA
CACTGACTCTACAGGCTCACCAGATCGAGATGGAATGCAGCCCAGCCCAC
ACGAGCTCCCCTACCATAGCAAAGCAGAGTGTGCCCGAGAAGGAGGGAAC
AAAGCTTCGAAGAAAAGCAATGGGGCACCAAATGGATTTTATGCGGAAAT
TGATTGGGAAAGATATAACTCACCTGAGCTGGATGAAGAAGGTTACAGCA
TCAGACCTGAGGAACCAGGCTCTACCAAAGGAAAGCACTTTTATTCTTCA
AGTGAATCCGAAGAGGAGGAAGAATCGCACAAGAAGTTCAATATCAAGAT
TAAACCCTTGCAGTCCAAGGACATCCTTAAGAATGCTGCAACAGTAGACG
AGCTGAAGGCTTCCATAGGCAACATTGCACTTTCCCCTTCGCCTGTGAGG
AAAAGTCCGAGGCGCAGCCCGGGTGCAATTAAAAGGAACTTATCCAGTGA
AGAAGTCGCAAGACCCAGGCGTTCCACCCCAACTCCAGAACTTACAAGCA
AGAAGCCTCTGGACGACACTCTGGCCCTTGCTCCCCTCTTTGGCCCACCG
TTAGAATCTGCTTTTGATGGACACAAGACGGAAGTTCTTTTAGATCAGCC
TGAGATATGGGGTTCAGGCCAACCAGTTAACCCAAGCATGGAGTCACCAA
AGCTAGCAAGACCTTTTCCCACTGGAACCCCTCCACCTCTGCCTCCAAAA
ACTGTACCAGCCACCCCGCCTCGGACAGGCTCCCCCTTAACAGTGGCGAC
AGGAAATGACCAGGCAGCCACAGAGGCCAAAATTGAGAAACTACCATCCA
TCAGTGACCTGGACAGCATTTTTGGCCCCGTGTTGTCCCCCAAGTCTGTT
GCTGTTAATACTGAGGAGACGTGGGTCCATTTCTCTGATGCATCCCCGGA
ACATGTTACTCCAGAGTTGACTCCAAGGGAAAAGGTGGTGACCCCACCAG
CTGCATCAGACATCCCAGCTGACTCCCCAACTCCAGGCCCGCCTGGCCCC
CCAGGCTCGGCAGGTCCCCCAGGGCCTCCTGGTCCTCGCAATGTACCATC
TCCGCTCAATTTAGAAGAAGTCCAGAAGAAAGTCGCTGAGCAGACCTTCA
TTAAAGATGATTACTTAGAAACACTCTCATCTCCTAAAGAGTGTGGGTTG
GGACAGAGAGCAACTCCACCTCCCCCACCACCACCCACCTACAGGACTGT
GGTTTCGTCCCCCGGACCTGGCTCGGGCAGTGGTACGGGGACCGCCAGTG
GTGCATCGTCCCCTGCTCGGCCAGCCACCCCCTTAGTTCCTTGCAGCTGC
TCCACTCCGCCTCCACCTCCTCCCCGGCCTCCATCCCGGCCAAAGCTACC
TCCAGGAAAGCCTGGAGTTGGAGACGTGTCCAGACCTTTTAGCCCACCCA
TACACTCCTCCAGCCCTCCTCCAATAGCACCCTTAGCCCGGGCTGAAAGC
ACTTCTTCAATATCATCAACCAATTCCCTGAGCGCAGCCACCACTCCCAC
AGTTGAGAATGAACAGSCTTCCCTCGTTTGGTTTGACAGAGGAAAGTTTT
ATTTGACTTTTGAAGGTTCTTCCAGGGGACCCAGTCCTCTAACTATGGGG
GCCCAGGACACCCTCCCGGTTGCAGCAGCATTCACAGAAACTGTCAATGC
CTACTTCAAAGGAGCAGATCCAAGCAAATGCATTGTTAAGATCACGGGAG
AAATGGTGTTGTCCTTTCCTGCTGGCATCACCAGACACTTTGCCAACAAC
CCATCCCCAGCTGCTCTGACTTTTCGAGTGATAAATTCCAGCAGGTTAGA
GCACGTCCTGCCGAACCCCCAGCTCCTCTGCTGCGATAACACACAAAATG
ATGCCAATACCAAGGAATTCTGGGTAAACATGCCAAATTTGATGACCCAC
CTGAAGAAGGTCTCTGAACAAAAACCCCAGGCTACATATTACAATGTGGA
CATGCTCAAGTATCAGGTGTCAGCCCAGGGCATTCAGTCCACACCTCTGA
ACTTGGCGGTGAACTGGCGCTGTGAGCCTTCCAGCACTGACCTGCGCATA
GATTATAAGTACAACACGGATGCCATGTCCACCGCAGTGGCCCTTAACAA
CGTGCAGTTCCTGGTCCCCATTGATGGAGGAGTGACCAAGCTCCAGGCTG
TCCTTCCTCCAGCAGTCTGGAATGCTGAACAACAAAGAATATTATGGAAG
ATTCCTGATATCTCCCAGAAGTCAGAAAATGGAGGCGTAGGTTCTTTACT
GGCAAGATTTCAATTAGCCGAAGGCCCAAGCAAACCTTCCCCACTGGTCG
TGCAGTTCACGAGTGAAGGGAGCACTCTGTCTGGCTGCGACATTGAGCTT
GTCGGAGCAGGGTACGGGTTTTCACTCATCAAGAAGAGGTTTGCTGCAGG
AAAATACTTGGCCGATAACTAATAAAATGTCATGCAAGGATTTTGAAGAT
CCATGTCCTGGAGAACTGTTGTCTGAGAGACATATTTTAATCTGGTTTGA
GGAAAACAAACCAACCGATGTCTGTACGTGGGCTCTGTCAGCTGGAAGGT
CCCGGCTTTCAGCCGTGATTTCCCACACCCAGTACAAGGAGGATCAGTTC
TACAGTACTTACTTCTAGGTGTACTATTGTTAATGGTTTTAAAATGTAAT
TATTGTATTTGTAAACTGTACCTTCATTCCAGTAAGGCAGTTAGACACCT
GAGTTTTAGCTTTTTTTTCCATTCCTGAAACGGATGTAATTTAAACTGCG
GTATGTAAATTTAATAGTAGTACTGTCGAATGGCACAATGCTTACAGAGA
TACAGTGCATTTTGTCAATATATAAAATTTAAATATAATGTTGATAGTTA
CCATAAAGGGGGTGCCACACATCAAGAACCTTAAATGGAACCAGAAACAA
GCAAGCAAACAAACAAACAAACAAACAAAACCTTACTTTTCTTCACTCCT
TATTACATTTTCCTCTAGAGCTAAAGAAACTTCTAGCTTCGGTTTAGTGG
GTTAAATTCAGAAACTATTTCAGAAAAAAAAAAAAATTCTGAAGTTACAG
CATATTCAAAGAGAAGCATTAATTACCACTTTTTTAAAAGCTTTTTTTTC
AAACCGCAAATTTCATAAAAATGCAAACTGTGTAAACAGGGCCTCTTATT
TTTATAACTTGTGTAAAAAGGGAAAATCAATTCATATTTAAAGTTTAAGT
AGTATTAAATTATATCCAAGAGTGAAGAGGATGTTGAAATCTTACCTGAC
CCCATGCCCCTTCTTTGCAGTTTAGCAAATGTTGAGATTGCTAAATCATC
AGATTAAAGCCAACTTGATTTTTAAAGTTTCAAGACTTTCTGAAGCTGAA
CTGGTTAAAACTTTTGCACAATTGCTTGGAACGGAGGGGGAGGGGCCTCT
CTGGTCCAGCACAGGTACCTTGTTTCTTCCCTACTCACAAGAATCAAAAC
AATGAAAGTCAAGAACCACAGAGGGGGGAAATTAGTTCCCTGTTCAGTCC
AAAAGGAGAACTTTAAACTTATCATTTACGTCTTTGGGGAAGGAAGAAAT
AAGCTTTATAAGTGAAATCCTATTCACCTTGTTGTCCTATGAATGTTTTC
GGGGTGACTTTAAGATTCATTGTATACATGTGCGAGTCTCTGCTATTCTT
GGGGAGTTGAAAGCAGAGCCAGGCCAGTGGCCTTGAAGTTCAGTAAATGC
CACAGTTCTGGGGCAAAGGTAGGCATGAGGGTTCTGCCCCTCAGCACAGG
AATCAGAGCAGTGTCTTGTAAGGTCTAAAGATTAAGTCTTCCAGTAAGCC
ACAAGTTATTTTGTAACAGAGTTGGGGAGTTTTGGCACTCGCTGCTGACT
TTCATTTTGTATCCACTCAAATGGAGTCTTCAACTCTTTTCAACTTTAGA
ATCAAATTAATTTTTTTTTTTTTTTTTTTTTTTTTACACAAGGTTTACTC
TGTGTAACTGTCCTGGATGTTCTGGAACTCTTTTTGTAGACCAGGCTGGC
CTCGAACTCAGAGAGATCCACCTGCCTGTGCTCCCCAAGTGCTGGGATTA
AAGGCGTGTGCCACCATGCCTGGCTTAGATTAAATTTTTTAAGTCTTACT
TCACCAGTGAGATTGTGATTGGCAGTTGTTTCGAGAGAGCTTTGTAGCTT
AATCTATGTTCTCTTCAATCAATGCTTGCTACCAAAAGAATGTCCAAAAT
GATCTATTTTTCCTGGGAACAATTCATCTATTTAAATAGGCTCTTGCCTA
GTTCCCCAAAGCAGCCTGTCTTTGAAGGTTTTTTTGAACAAAATAATTTT
TTCACAAAAAGTTTGGTTTTGAAATCAAAATAGAGAAATAAAATGTAAAT
TTTAAATCTAATGGAACATGAGGAAATGAAAAAACTTAAGCCAATGGAGA
GTAAAAGCAGAAAAAAATGAAACTTACCTAGAATGTGATTATATTATGTT
TTTAAGTAGTCAATTCATGGAAAAATATTGAATATTAACACAAAGCATAT
TAAAAATATGTAAATATTACTGTTTCTCATGTCTTTCTCTTTATATCTTA
TTTTATATAGTTTTAGAATGAATTGGTCATTAAATACAGTGTTTCTTTCC
AAAGAATAATTTTGTTGATATTGTAAAAATGTAATTAAAGATAGAGACTT
GAATAGTCTCTAACATTATCCAAATGTTTCTAGGAACCAAATTCAAAGCT
GTGAAGAAAGCTTGCAATCCCTGAATTGGCTTTTGTGAAATGGAATGACG
GTGGGTAATCTCAAAATTCAGACTTGAATAGTCAGAGCTGAAGTGGGGAA
TGGGTGGTTCCTTCTGGTTCAGAAAATAGGTCAAATAACAGCATTTGCTC
GCATCAGGGATGGAGATGTTGGTGATGTTTGGTTTTACTCTCGCAGGCTT
TCGTCTCCTGTTGAAGGTGTATCTGTAGCCCAGTGGGATAAGAGTTCATG
TTCTGAGATGTGGTCCTAGACAAGGCAGGCAAGGTTTCAGTCATCAATAC
CTATCAGGTCAGGTTCCCTTTTGTCTATACAAAATGGGTTAGCTCATAGC
CAGATGGTTTGCAGGACAGTGAGCTAAATTAGGACAAGATTCTGGTTAGC
CAAAGAGCTGTTTCCTAAGCACTCTGATTTTTTTTTAAAGCTGATAGAAA
GTGTAAATGTTCTATTTTGACGACATGGAAAGTATGTTTTCCTCTTCAAA
TAAATCCCTTATTTTTATGAAATTTTCAAAAATAAATTCTTGTTTAAAAT
AGTCTGAATGTTATCATAGTTGGAACTTGGCAATTACTAATTTGAAATTC
TATGAGATGTATCTCCAGCTAAAATGGCAATTCCCTGTATGCTATCTGGG
GCTCAGTTTACCTCTAAGGAAGACTGTCAGAGTGCAAATGGTTTTGAGTG
ACGGGAAAGTCAAAGGGCAAATGTTTGTGCTTTTTTCTTTTTCTGTCTTA
TATACTTCTTCTTGGTCTCAGAATGCAAAGTATCAGAGCCATAGTTACAC
ACATTTCCACTTTTAACGCTTCTTTTGAAGGAAGCAGATCCACTTTTGCC
CCGCCACTCATGCCTGCTGTGCAGACTCAGACGAGTCCCTGCCCTCTTCA
CGCCTTTGGGGTGAGAGGGGAGCCATATGTAAGTAGTTTTCAAGCTTTTC
TTAATGGGACTTTTCTTTTTCTAATAAAATCATGCCTGGAATCCTGTAAA
GATTGTTGCCTGGCTGTGAAGGGGCTTCTCCAGATCCTGAAATATAGCAT
CACAATACGTAAATGACTCCCGATGGATCTCCCAGCTCTGAAGACTTGCT
CTTCTACTTCACATGTGTAGCCACGACGATCAGCTGGCACACAGTACAAT
TAGCTGTGTAGTGAGTGCTCCCCAGCTATCAGTCATGAAACATATCACTT
TGCTCAACCTGTTTTTAAAAAAGCTCCAAAATGGTAAAAATGCTTTTCAG
TCTTTGTTTTCCCAATAATGGTATTGAGGCCTAAGCTGATTAACTTCCCC
CAAAGTGGTACCACAGCTGGTAACGACCCCAATGATCCTGAAAAAAATGG
AATGAGTACCTTGCTGTTTCRTTTAGTTYATTTTGGGAAAATAATCCATT
TGAATGTCAAGATAAAAAGGCACCAGGAAAAGTCCTCATTGGAAGGATTA
AAGATGAGCCTGGTAAGATGTTAAGATGTAAGATGTTAAGATGTGTTACT
GTAAAAAAAAAAAGCTT.

The corresponding amino acid sequence is as follows:—

[SEQ ID NO:4]

MMEGLKKRTRKAFGIRKKEKDTDSTGSPDRDGMQPSPHELPYHSKAECAR
EGGNKASKKSNGAPNGFYAEIDWERYNSPELDEEGYSIRPEEPGSTKGKH
FYSSSESEEEEESHKKFNIKIKPLQSKDILKNAATVDELKASIGNIALSP
SPVRKSPRRSPGAIKRNLSSEEVARPRRSTPTPELTSKKPLDDTLALAPL
FGPPLESAFDGHKTEVLLDQPEIWGSGQPVNPSMESPKLARPFPTGTPPP
LPPKTVPATPPRTGSPLTVATGNDQAATEAKIEKLPSISDLDSIFGPVLS
PKSVAVNTEETWVHFSDASPEHVTPELTPREKVVTPPAASDIPADSPTPG
PPGPPGSAGPPGPPGPRNVPSPLNLEEVQKKVAEQTFIKDDYLETLSSPK
ECGLGQRATPPPPPPPTYRTVVSSPGPGSGSGTGTASGASSPARPATPLV
PCSCSTPPPPPPRPPSRPKLPPGKPGVGDVSRPFSPPIHSSSPPPIAPLA
RAESTSSISSTNSLSAATTPTVENEQASLVWFDRGKFYLTFEGSSRGPSP
LTMGAQDTLPVAAAFTETVNAYFKGADPSKCIVKITGEMVLSFPAGITRH
FANNPSPAALTFRVINSSRLEHVLPNPQLLCCDNTQNDANTKEFWVNMPN
LMTHLKKVSEQKPQATYYNVDMLKYQVSAQGIQSTPLNLAVNWRCEPSST
DLRIDYKYNTDAMSTAVALNNVQFLVPIDGGVTKLQAVLPPAVWNAEQQR
ILWKIPDISQKSENGGVGSLLARFQLAEGPSKPSPLVVQFTSEGSTLSGC
DIELVGAGYGFSLIKKRFAAGKYLADN.

### EXAMPLE 9

### AGT-121 Taqman Tissue Distribution

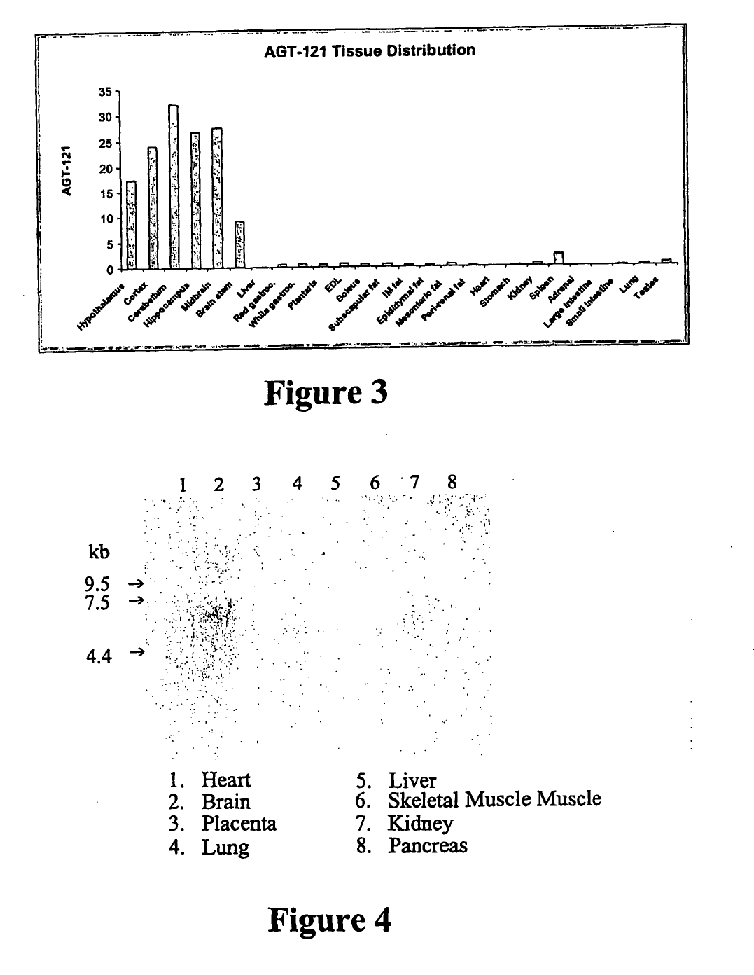

Tissue distribution of AGT-121 was investigated by Taqman PCR in multiple tissues of P. obesus (FIG. 3). Highest levels were seen in the brain with very low levels also evident in the spleen.

### EXAMPLE 10

### AGT-121—Clontech MTN Human RNA Blot

Tissue distribution of AGT-121 in human tissues was also examined by Northern analysis of a Clontech multiple tissue RNA blot. A specific band of approximately 6 kb was seen in the brain (FIG. 4). AGT-121 is thought to be brain-specific.

### EXAMPLE 11

### AGT-121 Alleles are Associated with Obesity

The insertion and deletion alleles described are associated with obesity. Eighty lean and obese individual Psammomys obesus were genotyped for the presence of the deletion, or the insertion, or both. Diabetic animals were not considered. The genetoype is significantly associated with the obesity phenotype seen in P. obesus. The results are shown in Tables 4 and 5.

TABLE 4
Summary of results

Phenotype

| | | Lean | Obese | Total |
|---|---|---|---|---|
| Genotype | Insertion | 24 | 9 | 33 |
| | Heterozygote | 17 | 15 | 32 |
| | Deletion | 5 | 10 | 15 |

| | | | |
|---|---|---|---|
| Total | 46 | 34 | 80 |

TABLE 5
Chi squared tests

| | | | Asymp. Sig. |
|---|---|---|---|
| | Value | df | (2-sided) |
| Pearson Chi Square | 6.967 | 2 | .031 |
| Likelihood Ratio | 7.092 | 2 | 0.29 |
| Linear-by-Linear Association | 6.879 | 1 | .009 |
| No. of valid cases | 80 | | |

### EXAMPLE 12

### AGT-121 Gene Expression in Energy Restricted Hypothalamus

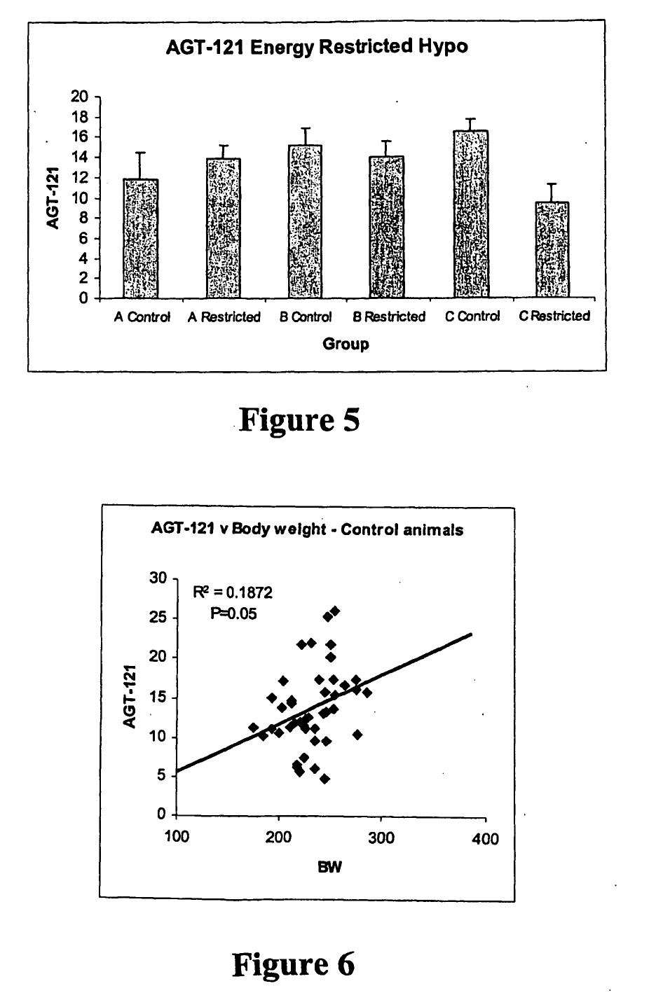

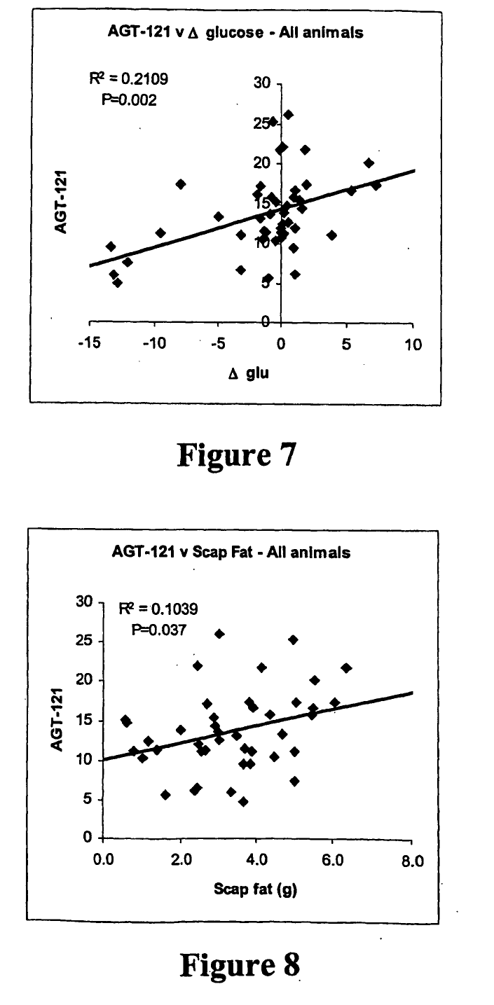

Oligonuclotide primers were designed in the coding sequence of Psammomys obesus AGT-121. Expression of AGT-121 was analyzed in energy restricted hypothalamus. Positive correlations were seen with body weight in control animals, change in glucose in all animals and subscapular fat mass in all animals (FIGS. 5-8).

### EXAMPLE 13

### AGT-121 Sequence Homology

The AGT-121: The ISR protein shows strong homology at both the nucleotide and protein level to human hypothetical protein DKFZp761D221 (DKFZp761D221) [Accession: NP_{—}115667]. This protein is predicted to contain the pfam00928.5, Adap_comp_sub domain which is identified as adaptor complexes medium subunit family. This family also contains members which are cocatomer subunits. This gene has been localized to human chromosome 1p31.2.

### EXAMPLE 14

### Partial Sequence of Psammomys obesus AGT-122

AGT-122 was identified using differential display PCR of liver cDNA from diabetic and non-diabetic Psammomys obesus.

The partial nucleotide sequence is as follows:

[SEQ ID NO:5]

TCGCGGATCCAGACGCTGCGTTTGCTGGCTTTGATGAAATTTTTAATTTT
TCAATATCAGGAATGTTTAACTATGCCATGAATTTATGGTAGTCAAGGTT
GGAAGGCAGGGGAGAGGACACAGGGAGTAAAGGCACTTGCCCCCAAGCCT
TACAACCTGAATTCCATCCCAGAGTGCCTAATGGTTGAAGGACGGAACTG
AATATCTCTAGCTGTCCTCTATCCTCCACAGATACACAGTGAATGCATCA
ACGTAAAAAATTACAGCTAGAAATAATGTCGTGCCATTCATTGTATTTTA
CATTNGTNCATCTTNGNTTTTCCATANTAAAAATGTCTNAGACATACCAC
TTAAAAAAAAAAGCTT.

### EXAMPLE 15

### AGT-122 Gene Expression

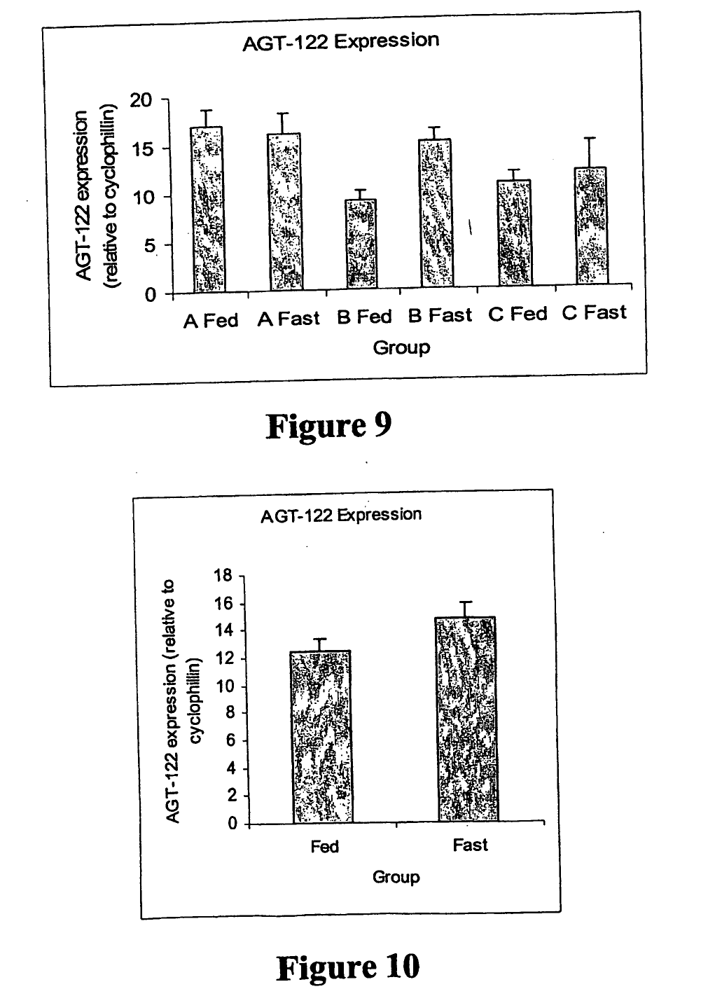

There was a significant difference in AGT-122 gene expression in the fed state between Group A compared to Group B (p=0.001) and Group C (p=0.005) animals (FIG. 9). In the fasted state there were no significant differences between the groups. Within the groups of animals, significantly increased expression was seen only in the Group B fasted animals (p=0.009). When data from all groups were pooled, no significant differences were seen (FIG. 10).

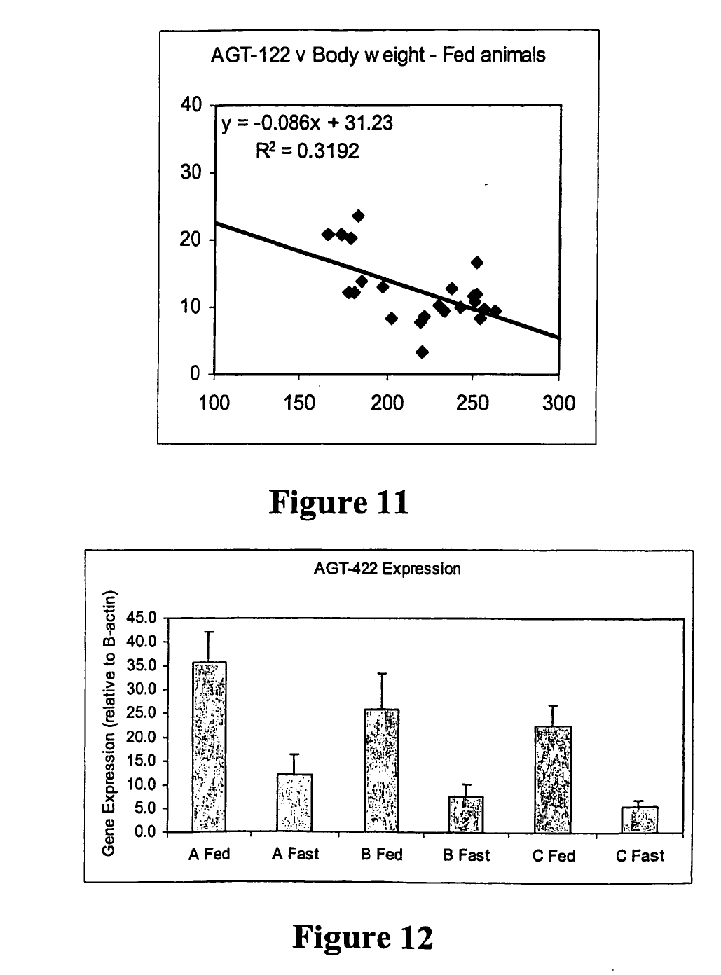

Expression of AGT-122 in grouped fasted animals showed no association with body weight, glucose, or insulin. Expression of AGT-122 in fed animals showed a significant negative correlation with body weight (p=0.005; FIG. 11), glucose (p=0.003) and insulin (p=0.015).

### EXAMPLE 16

### AGT-122 Sequence Homology

The AGT-122 sequence shows significant homology with a number of regions on different mouse chromosomes. There was no homologous sequence on the public database (BLASTN version 2.2.1).

### EXAMPLE 17

### Partial Sequence of Psammomys obesus AGT-422

AGT-422 was identified by Suppression Subtractive Hybridization (SSH) [also referred to as Representational Difference Analysis (RDA)] of liver cDNA from diabetic and non-diabetic Psammomys obesus.

The partial nucleotide sequence is as follows:

[SEQ ID NO:6]

CCCTATCCGCTACCCCTGGGAGGGACACAAAAACACATTTTGTGTTTTTG
AAAAAACTGAGGTCACCAGACTCTTGTATTGTCTTCTTGGACTTCTCTCA
GGAACACTCAGGACTCTCCCCACACAACACCGTTCTTGAACCGTTCTAAC
AAATGTTTAAAGTGGTTTCCTTTGAACCACATTAAATTTAGTTTAAGCAG
TCACCAGTGGGCTAGCAGTTCTGGGTTGGGCAGCACATCTTGTACAAGCT
CTTCCATCTGCCAGGATCACCACCTCTCTGACTTGCACATTTGTGGGTTC
CCCACAGACGAATGGGATGAGTGAAAGAGTGAGTATGTTCTGTTGGGCCT
TCAGTAACAGAAGACTGATTCAGAAAGTAGCACACGTCACATTTTTCTGT
AGGTTGGTTTGTTTAGTTTCATTTTTGATTTGTGGAACAAAA.

### EXAMPLE 18

### AGT-422 Gene Expression

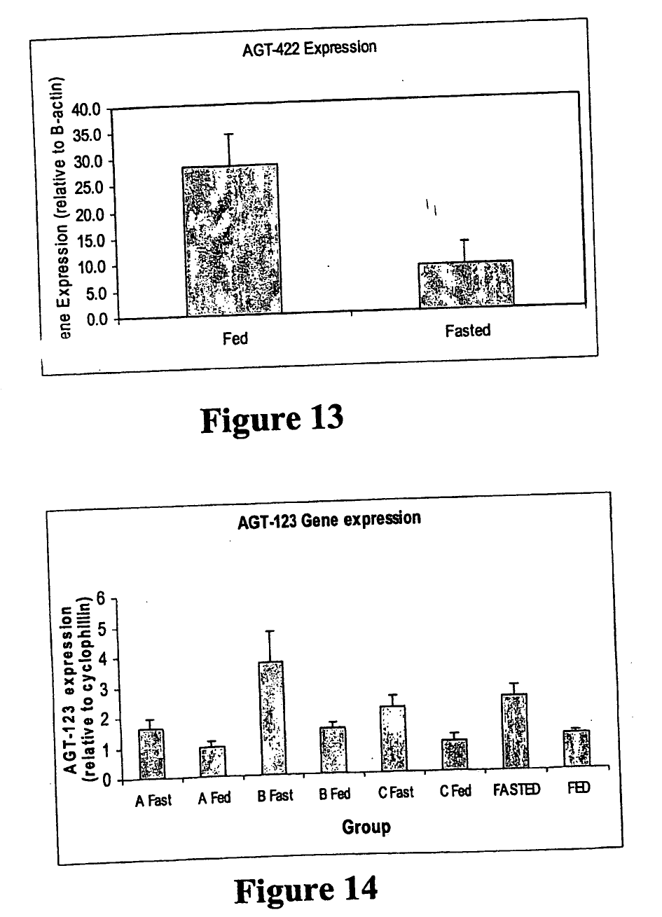

AGT-422 was normally distributed. One way ANOVA with an LSD post hoc test found gene expression tended to be higher in fed Group A animals than fed Group C (p=0.068). Gene expression was significantly greater in fed Group A animals than fasted Group A (p=0.002), fed Group B animals than fasted Group B (p=0.014) and fed group C animals than fasted group C (p=0.039) [FIG. 12]. An independent samples T test found AGT-422 gene expression greater in fed animals than fasted animals when group data were combined (p<0.001; FIG. 13).

### EXAMPLE 19

### AGT-422 Sequence Homology

The AGT-422 sequence shows sequence homology to Rattus norvegicus clone CH230-213K1. The full gene and open reading frame are yet to be identified and a human homolog is also as yet undetermined.

### EXAMPLE 20

### Partial Sequence of Psammomys obesus AGT-123

AGT-123 was identified using differential display PCR of hypothalamic cDNA from diabetic and non-diabetic Psammomys obesus.

The partial nucleotide sequence is as follows:

[SEQ ID NO:7]

TGAGAGTCCATCTCAGCTTATTTCATTGAGATGTTTTGATTAAGAAGTAT
GACTAGATTAAAAAATTCTATATAGCTTGGCATTGTTGATAGTTTATATA
TTTCACTGATTTCTGGTCCCTTGAAAGTTACTTGGTGATCAACATAGTGT
AGTGAAAGGATTGGGATGGACATTAAAAAAAAAAAGCTT

### EXAMPLE 21

### AGT-123 Gene Expression

Hypothalamic AGT-123 expression tended to be increased with fasting in Group A animals (p=0.06) and was significantly increased with fasting in Group B and Group C animals (p=0.02 and p=0.01, respectively) [FIG. 14]. When all fed and fasted animals were combined, the fasted animals had significantly increased expression compared to fed animals (p=0.001) [FIG. 14]. AGT-123 expression was not correlated with glucose or insulin concentrations. There was a tendency for a relationship between AGT-123 expression and body weight, although this was not statistically significant (p=0.08).

### EXAMPLE 22

### AGT-123 Sequence Homology

The AGT-123 sequence matches three mouse expressed sequence tags (gb|BG797393.1|BG797393 ic14h03.x1 Kaestner ngn3 wt Musi musculus; gb|AI661150.1|AI661150 va01a02.x1 Soares mouse lymph node NbMLN; dbj|BB257743.1|BB257743 BB257743 RIKEN full-length enriched, 7 d) however, the corresponding gene has not yet been identified (BLASTN version 2.2.1).

### EXAMPLE 23

### Partial Sequence of Psammomys obesus AGT-504

AGT-504 was identified using Amplified Fragment Length Polymorphism (AFLP) screening of genomic DNA from diabetic and non-diabetic Psammomys obesus. It was found to be expressed in the liver of diabetic and non-diabetic Psammomys obesus.

The partial nucleotide sequences are as follow:

Genomic DNA:-

[SEQ ID NO:8]

TGGNTACTCTTGNAAGCACCTTGAAAGTTCAGCCTGCTAACTTACCTTTT
TAGCTTTAGATTGCTTAGATATTCAAATGAGAGTGTGGTGCAAATCCTGA
TTACAAGGAGTTTGAGTTTGGAGTGATTGGCAATGCACTTGTGAGGCTGT
GTGCCTATGGTCCTAGGACTTAGGAGGCAGGGATAGAAGGACCAGGTGCT
GAAAGACAGCCTTGGCTAGTTAGTGGACAGATACATAAATGTACTGCATG
AGATTCTTTCAGAATAACAACCTCCTTTTAAAGAAGTTACTTCTGACATG
GAATCTGTTGCCTGCTTTTGGATCACTACCCCCTGGTGGGACACCTTTGC
CAGACCATGGAGGAAGAACTGTCTTGAT.
cDNA:
AACGTGAGCTTTTTGGAAGGCCCAGANAATTTAAGGAAAGTGTCCCGGCA
AATCCTGATTACAAGGAGTTTGAGTTTGGAGTGATTGGCAATGCACTTGT
GAGGCTGTGTGCCTATGGTCCTAGGACTTAGGAGGCAGGGATAGAAGGAC
CAGGTGCTGAAAGACAGCCTTGGCTAGTTAGTGGACAGATACATAAATGT
ACTGCATGAGATTCTTTCAGAATAACAACCTCCTTTTAAAGAAGTTACTT
CTGACATGGAATCTGTTGCCTGCTTTTGGATCACTACCCCCTGGTGGGAC
ACCTTTGCCAGACCATGGAGGAAGAACTGTCTTGATGGGANN

### EXAMPLE 24

### AGT-504 Gene Expression

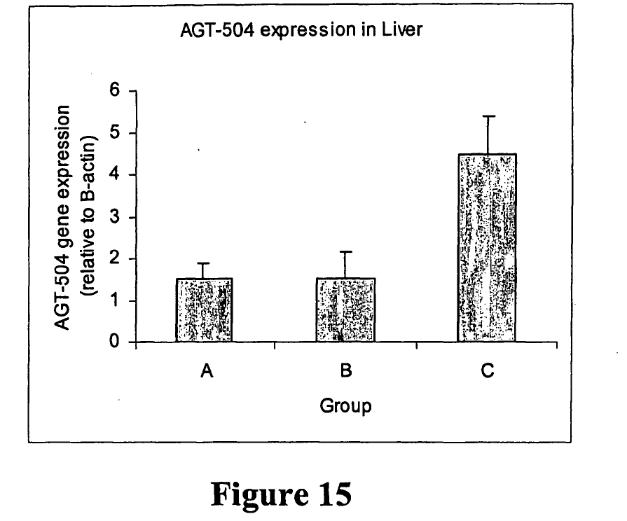

Hepatic AGT-504 expression was normally distributed and ANOVA with LSD post hoc analysis showed that Group C animals had significantly higher AGT-504 gene expression compared to both Group A (p=0.014) and Group B (p=0.02) animals (FIG. 15).

### EXAMPLE 25

### Amplified Fragment Length Polymorphism (AFLP) Technique

The AFLP technique is based on the amplification of subsets of genomic restriction fragments using polymerase chain reaction (PCR). DNA is cut with restriction enzymes (EcoRI and MseI) and double-stranded adapters are ligated to the ends of the DNA fragments to generate template DNA for amplification. The sequences of adapters and the adjacent restriction site serve as primer binding sites for subsequent amplification of the restriction fragments. Selective nucleotides are included at the 3′ ends of the PCR primers, which therefore prime DNA amplification only from a subset of the restriction fragments. This method will identify sequence differences in the restriction sites that are associated with the obesity/diabetes phenotype.

AFLP was performed using the GibcoBRL “AFLP Analysis System I” and “AFLP Starter Primer” Kits according to the manufacturer's instructions.

Six genomic DNA pools of each group of Psammomys obesus (n=15) were used in an AFLP screen of 256 primer pairs, which equates to a genomic scan at a density of approximately 1.4 cM. The animals were divided by sex and then into three groups corresponding to lean (Group A), obese (Group B) and obese/diabetic (Group C).

The PCRs were performed in 20 μl under the following conditions: one cycle at 94° C. for 30 s; 65° C. for 30 s; and 72° C. for 60 s, then for each consecutive cycle the annealing temperature was lowered by 0.7° C. for 12 cycles. This gives a touchdown phase of 13 cycles. This was followed by 23 cycles at 94° C. for 30 s, 56° C. for 30 s and 72° C. for 60 s.

Once amplified, the fragments were separated on a 6% (w/v) polyacrylamide gel to visualize the (typically) 50-150 bands. Those bands that were deemed to be different between the groups of animals were excised from the gel. Re-amplification of the band of interest was performed using the following PCR conditions: 94° C. 2 min, 40 cycles of (94° C. 30 s; 54° C. 30 s; 72° C. 60 s); 72° C. 5 min and the following primers: F 5′-GTA GAC TGC GTA CCA ATT C-3′ [SEQ ID NO:10] and R 5′-GAC GAT GAG TCC TGA GTA A-3′ [SEQ ID NO:11]. The amplified bands were sequenced with Applied Biosystems Big Dye sequencing kit.

### EXAMPLE 26

### Primers

Primer and probe sequences for amplification and analysis of each gene (shown in the 5′ to 3′ direction).

| SYBR Green analysis | | |
|---|---|---|
| AGT-119 | | |
| Set 1 | | |
| Forward: | ggattatcgtgaccagataggagc | [SEQ ID NO:12] |
| Reverse: | acgcatcttgttgggctaatg | [SEQ ID NO:13] |
| Set2 | | |
| Forward: | cgcaaccaaattagccagtg | [SEQ ID NO:14] |
| Reverse: | gcatcttgttgggctaatgacat | [SEQ ID NO:15] |
| AGT-120 | | |
| Forward: | acccagatgctggctgact | [SEQ ID NO:16] |
| Reverse: | ctggccaccagtcagtgaataa | [SEQ ID NO:17] |
| AGT-121 | | |
| Forward (insertion): | aaaacatgagagaagccatactaattca | [SEQ ID NO:18] |
| Forward (deletion): | cacaaaacatgagatactaattcataagtga | [SEQ ID NO:19] |
| Reverse: | tgaaaccaagatagcacaaacgaa | [SEQ ID NO:20] |
| AGT-122 | | |
| Forward: | catcccagagtgcctaatggtt | [SEQ ID NO:21] |
| Reverse: | acgttgatgcattcactgtgtatct | [SEQ ID NO:22] |
| AGT-422 | | |
| Forward: | cagtcaccagtgggctagca | [SEQ ID NO:23] |
| Reverse: | cctggcagatggaagagctt | [SEQ ID NO:24] |
| AGT-123 | | |
| Forward: | gcttggcattgttgatagtttatatatttc | [SEQ ID NO:25] |
| Reverse: | cactacactatgttgatcaccaagtaactt | [SEQ ID NO:26] |
| AGT-504 | | |
| Forward: | gaatctgttgcctgcttttgg | [SEQ ID NO:27] |
| Reverse: | ctccatggtctggcaaaggt | [SEQ ID NO:28] |
| Taqman analysis | | |
| β-actin Forward: | gcaaagacctgtatgccaacac | [SEQ ID NO:29] |
| β-actin Reverse: | gccagagcagtgatctctttctg | [SEQ ID NO:30] |
| Probe: | FAM-tccggtccacaatgcctgggaacat-TAMRA | [SEQ ID NO:31] |
| Cyclophilin Forward: | cccaccgtgttcttcgaca | [SEQ ID NO:32] |
| Cyclophilin Reverse: | ccagtgctcagagcacgaaa | [SEQ ID NO:33] |
| Probe: | FAM-cgcgtctccttcgagctgtttgc-TAMRA | [SEQ ID NO:34] |

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Altschul et al., Nucl. Acids Res. 25: 3389, 1997.
Australian Institute of Health and Welfare (AIHW), Heart, Stroke and Vascular diseases, Australian facts. AIHW Cat. No. CVD 7 Canberra: AIHW and the Heart Foundation of Australia, 1999.
Ausubel et al., “Current Protocols in Molecular Biology” John Wiley & Sons Inc, Chapter 15, 1994-1998.
Barnett et al., Diabetologia 37: 671-676, 1994a.
Barnett et al., Int. J. Obesity 18: 789-794, 1994b.
Barnett et al., Diabete Nutr. Metab. 8: 42-47, 1995.
Bonner and Laskey, Eur. J. Biochem. 46: 83, 1974.
Bouchard, The genetics of obesity. Boca Raton: CRC Press, 1994.
Collier et al., Ann. New York Acad. Sci. 827: 50-63, 1997a.
Collier et al., Ex.p Clin. Endocrinol. Diabetes 105: 36-37, 1997b.
De Looper, M and Bhatia K, Australia's Health Trends 2001. Australian Institute of Health and Welfare (AIHW) Cat. No. PHE 24. Canberra: AIHW, 2001.
Douillard and Hoffman, Basic Facts about Hybridomas, in Compendium of Immunology Vol. II, ed. by Schwartz, 1981.
Kohler and Milstein, Nature 256: 495-499, 1975.
Kohler and Milstein, European Journal of Immunology 6: 511-519, 1976.
Koopmans, Experimental studies on the control of food intake. In: Handbook of Obesity, Eds. GA Bray, C Bouchard, WPT James pp 273-312, 1998.
Kopelman et al., Int. J. Obesity 18: 188-191, 1994.
Kopelman, Nature 404: 635-643, 2000.
Marmur and Doty, J. Mol. Biol. 5: 109, 1962.
Mokdad et al., JAMA. 282(16). 1519-1522, 1999.
Mokdad, Diabetes Care 24(2): 412, 2001. Must et al., JAMA. 282(16): 1523-1529, 1999.
National Health and Medical Research Council, Acting on Australia's weight: A strategy for the prevention of overweight and obesity. Canberra: National Health and Medical Research Council, 1996.
Ravussin, Metabolism 44(Suppl 3): 12-14, 1995.
Russek, M., A hypothesis on the participation of hepatic glucoreceptors in the control of food intake. Nature 200: 176, 1963.
Shafrir and Gutman, J. Basic Clin. Physiol. Pharm. 4: 83-99, 1993.
Stellar, Psychol. Rev. 61: 5-22, 1954.
Walder et al., Obesity Res. 5: 193-200, 1997a.
Wolf and Colditz, Obes. Res. 6: 97-106, 1998.
Zimmet, Diabetes Care 15(2): 232-247, 1992.

1. A isolated nucleic acid molecule comprising a nucleic acid sequence selected from the group consisting of:
a. a nucleic acid sequence having at least 30% identity to a nucleic acid sequence selected from the group consisting of SEQ ID NO:1, SEQ ID NO:2, SEQ ID NO:3, SEQ ID NO:5, SEQ ID NO:6, SEQ ID NO:7, SEQ ID NO:8, and SEQ ID NO:9;
b. a nucleic acid sequence that hybridizes under stringent hybridization conditions with a nucleic acid sequence selected from the group consisting of SEQ ID NO:1, SEQ ID NO:2, SEQ ID NO:3, SEQ ID NO:5, SEQ ID NO:6, SEQ ID NO:7, SEQ ID NO:8, and SEQ ID NO:9; and
c. a nucleic acid sequence fully complementary to a nucleic acid sequence of (a) or (b); wherein said nucleic acid molecule of (a), (b), or (c) encodes an expression product that is differentially expressed in obese animals compared to lean animals, in fasted animals compared to fed animals, or in diabetic animals compared to non-diabetic animals in a tissue selected from the group consisting of the stomach, liver and hypothalamus.

2. The isolated nucleic acid molecule of claim 1 wherein the nucleic acid molecule comprises a nucleotide sequence selected from the group consisting of SEQ ID NO:1, SEQ ID NO:2, SEQ ID NO: 3, SEQ ID NO:5, SEQ ID NO:6, SEQ ID NO:7, SEQ ID NO:8, and SEQ ID NO:9, or a nucleic acid sequence fully complementary to any of said nucleic acid sequences.

3. The isolated nucleic acid molecule of claim 1 wherein the nucleic acid molecule encodes a protein comprising an amino acid sequence SEQ ID NO:4.

4. A therapeutic composition comprising a nucleic acid molecule of claim 1 and a pharmaceutically acceptable carrier or diluent

5. An isolated protein encoded by a nucleic acid sequence selected from the group consisting of:
a. a nucleic acid sequence having at least 30% identity to a nucleic acid sequence selected from the group consisting of SEQ ID NO:1, SEQ ID NO:2, SEQ ID NO:3, SEQ ID NO:5, SEQ ID NO:6, SEQ ID NO:7,
SEQ ID NO:8, and SEQ ID NO:9;
b. a nucleic acid sequence that hybridizes under stringent hybridization conditions with a nucleic acid sequence selected from the group consisting of SEQ ID NO:1, SEQ ID NO:2, SEQ ID NO:3, SEQ ID NO:5, SEQ ID NO:6, SEQ ID NO:7, SEQ ID NO:8, and SEQ ID NO:9; and
c. a nucleic acid sequence fully complementary to a nucleic acid sequence of (a) or (b); wherein said protein is differentially expressed in obese animals compared to lean animals, in fasted animals compared to fed animals, or in diabetic animals compared to non-diabetic animals in a tissue selected from the group consisting of the stomach, liver and hypothalamus.

6. The isolated protein of claim 5, wherein the protein comprises an amino acid sequence SEQ ID NO:4.

7. The isolated protein of claim 5, wherein the protein is encoded by a nucleic acid molecule comprising a nucleic acid sequence selected from the group consisting of SEQ ID NO:1, SEQ ID NO:2, SEQ ID NO:3, SEQ ID NO:5, SEQ ID NO:6, SEQ ID NO:7, SEQ ID NO:8, and SEQ ID NO:9.

8. A therapeutic composition comprising a protein of claim 5 and a pharmaceutically acceptable carrier or diluent.

9. An isolated antibody that selectively binds to a protein of claim 5.

10. A therapeutic composition comprising an antibody of claim 9 and a pharmaceutically acceptable carrier or diluent.

11. A method for modulating expression of a biological molecule selected from the group consisting of AGT-119, AGT-120, AGT-121, AGT-122, AGT-422, AGT-423 and AGT-504 in a mammal, said method comprising administering to said mammal an effective amount of a modulator of said biological molecule.

12. A method of modulating activity of a biological molecule selected from the group consisting of AGT-119, AGT-120, AGT-121, AGT-122, AGT-422, AGT-423 and AGT-504 in a mammal, said method comprising administering to said mammal an effective amount of a modulator of said biological molecule.

13. A method of treating a mammal suffering from a condition characterized by symptoms selected from the group consisting of obesity, anorexia, diabetes and energy imbalance, said method comprising administering to said mammal a composition capable of modulating the expression or activity of a biological molecule selected from the group consisting of AGT-119, AGT-120, AGT-121, AGT-122, AGT-422, AGT-423 and AGT-504.

14. A method of treating a mammal suffering from a condition characterized by symptoms selected from the group consisting of obesity, anorexia, diabetes and energy imbalance, said method comprising administering to said mammal a biological molecule selected from the group consisting of AGT-119, AGT-120, AGT-121, AGT-122, AGT-422, AGT-423 and AGT-504 or AGT-119, AGT-120, AGT-121, AGT-122, AGT-422, AGT-423 and AGT-504.

15. A method of detecting a compound capable of effecting the activity of a biological molecule selected from the group consisting of AGT-119, AGT-120, AGT-121, AGT-122, AGT-422, AGT-423 and AGT-504 or AGT-119, AGT-120, AGT-121, AGT-122, AGT-422, AGT-423 and AGT-504, said method comprising contacting said biological molecule with a candidate compound and observing the effect on the activity of said biological molecule.

16. A method of detecting a compound capable of effecting the expression of a biological molecule selected from the group consisting of AGT-119, AGT-120, AGT-121, AGT-122, AGT-422, AGT-423 and AGT-504 or AGT-119, AGT-120, AGT-121, AGT-122, AGT-422, AGT-423 and AGT-504, said method comprising administering a canditate compound to a mammal and observing the effect on the expression of said biological molecule.

17. A composition comprising a modulator of the expression or activity of a biological molecule selected from the group consisting of AGT-119, AGT-120, AGT-121, AGT-122, AGT-422, AGT-423 and AGT-504 and a pharmaceutically acceptable carrier or diluent.

18. A method for detecting a biological molecule selected from the group consisting of AGT-119, AGT-120, AGT-121, AGT-122, AGT-422, AGT-423 and AGT-504 in a biological sample, said method comprising contacting said biological sample with an antibody specific for any of said biological molecules and detecting a complex between said biological molecule and said antibody.

19. A isolated nucleic acid molecule comprising a nucleic acid sequence selected from the group consisting of SEQ ID NO:1, SEQ ID NO:2, SEQ ID NO:3, SEQ ID NO:5, SEQ ID NO:6, SEQ ID NO:7, SEQ ID NO:8, and SEQ ID NO:9.

20. An isolated protein encoded by a nucleic acid sequence selected from the group consisting of SEQ ID NO:1, SEQ ID NO:2, SEQ ID NO:3, SEQ ID NO:5, SEQ ID NO:6, SEQ ID NO:7, SEQ ID NO:8, and SEQ ID NO:9.


SEQUENCE LISTING (34 sequences)

SEQ ID NO: 1 - DNA, 181 bp, Psammomys obesus
  misc_feature (48)..(50): N = any nucleotide
aatgaaagaattgattgatacgcaaccaaattagccagtgaggttagnnncnggattatcgtgaccagataggagccttggaaaatgactaagaaaaatgaaaaacagcctaaaatgtcattagcccaacaagatgcgttaaaacgcctggatcaagttagaangcagaaaagcgaaagcc

SEQ ID NO: 2 - DNA, 814 bp, Psammomys obesus
  misc_feature (374)..(374): n = any nucleotide
gctgatggcgatgaatgaacactgcgtttgctggccttatcaattcccgctttttcttggatgaaaaagactaaccatgatcgggcccaacggcggaagtcgcttttgtcaccagtagtgatggtgccgggatcaagtgccacgattgagcgtttcaattcactgattgcgatgcttaataaagattcgccgcaccctcacagtgtgttaaagattaaagtcatgaaagatggcagtttaaaatacaagggcagcattaatcgcggtgataatgaaccctttattgtgattggttttgaaaataataaagatggctatagtaatattaagaagcaagcaagctggctagatattgccttttatgagatctcgcnaacttataaatttaacaactttaaggcctttggccattcaaatggagggctggtgtggacatattggttagagcattattattcagagtatgagtcagaaatcaaaatcaagcggttgatgactttggcttcaccatttaactttgacgaagacaatctgaatcaccggacccagatgctggctgactttattaaatatcggaaacgacttccaaaaacgctcaaagtttattcactgactggtggccagacctatgaatctgacgggattgttcctgaaaatagtgtagccgcagccaagtatattttccaaaatcaagtgaagagctttatggaaattacggttacgggtaagcagctaatcactcagatttaccgcaaaatgaacaagtagtgctagtgatgaatccaccactcactaaagataataaaaaaaaaaa

SEQ ID NO: 3 - DNA, 6317 bp, Psammomys obesus
cagactccttggaaattaaggaatgcaattctgccaccatgatggaaggactgaaaaaacgtacaaggaaggcctttggaatacggaagaaagaaaaagacactgactctacaggctcaccagatcgagatggaatgcagcccagcccacacgagctcccctaccatagcaaagcagagtgtgcccgagaaggagggaacaaagcttcgaagaaaagcaatggggcaccaaatggattttatgcggaaattgattgggaaagatataactcacctgagctggatgaagaaggttacagcatcagacctgaggaaccaggctctaccaaaggaaagcacttttattcttcaagtgaatccgaagaggaggaagaatcgcacaagaagttcaatatcaagattaaacccttgcagtccaaggacatccttaagaatgctgcaacagtagacgagctgaaggcttccataggcaacattgcactttccccttcgcctgtgaggaaaagtccgaggcgcagcccgggtgcaattaaaaggaacttatccagtgaagaagtcgcaagacccaggcgttccaccccaactccagaacttacaagcaagaagcctctggacgacactctggcccttgctcccctctttggcccaccgttagaatctgcttttgatggacacaagacggaagttcttttagatcagcctgagatatggggttcaggccaaccagttaacccaagcatggagtcaccaaagctagcaagaccttttcccactggaacccctccacctctgcctccaaaaactgtaccagccaccccgcctcggacaggctcccccttaacagtggcgacaggaaatgaccaggcagccacagaggccaaaattgagaaactaccatccatcagtgacctggacagcatttttggccccgtgttgtcccccaagtctgttgctgttaatactgaggagacgtgggtccatttctctgatgcatccccggaacatgttactccagagttgactccaagggaaaaggtggtgaccccaccagctgcatcagacatcccagctgactccccaactccaggcccgcctggccccccaggctcggcaggtcccccagggcctcctggtcctcgcaatgtaccatctccgctcaatttagaagaagtccagaagaaagtcgctgagcagaccttcattaaagatgattacttagaaacactctcatctcctaaagagtgtgggttgggacagagagcaactccacctcccccaccaccacccacctacaggactgtggtttcgtcccccggacctggctcgggcagtggtacggggaccgccagtggtgcatcgtcccctgctcggccagccacccccttagttccttgcagctgctccactccgcctccacctcctccccggcctccatcccggccaaagctacctccaggaaagcctggagttggagacgtgtccagaccttttagcccacccatacactcctccagccctcctccaatagcacccttagcccgggctgaaagcacttcttcaatatcatcaaccaattccctgagcgcagccaccactcccacagttgagaatgaacagscttccctcgtttggtttgacagaggaaagttttatttgacttttgaaggttcttccaggggacccagtcctctaactatgggggcccaggacaccctcccggttgcagcagcattcacagaaactgtcaatgcctacttcaaaggagcagatccaagcaaatgcattgttaagatcacgggagaaatggtgttgtcctttcctgctggcatcaccagacactttgccaacaacccatccccagctgctctgacttttcgagtgataaattccagcaggttagagcacgtcctgccgaacccccagctcctctgctgcgataacacacaaaatgatgccaataccaaggaattctgggtaaacatgccaaatttgatgacccacctgaagaaggtctctgaacaaaaaccccaggctacatattacaatgtggacatgctcaagtatcaggtgtcagcccagggcattcagtccacacctctgaacttggcggtgaactggcgctgtgagccttccagcactgacctgcgcatagattataagtacaacacggatgccatgtccaccgcagtggcccttaacaacgtgcagttcctggtccccattgatggaggagtgaccaagctccaggctgtccttcctccagcagtctggaatgctgaacaacaaagaatattatggaagattcctgatatctcccagaagtcagaaaatggaggcgtaggttctttactggcaagatttcaattagccgaaggcccaagcaaaccttccccactggtcgtgcagttcacgagtgaagggagcactctgtctggctgcgacattgagcttgtcggagcagggtacgggttttcactcatcaagaagaggtttgctgcaggaaaatacttggccgataactaataaaatgtcatgcaaggattttgaagatccatgtcctggagaactgttgtctgagagacatattttaatctggtttgaggaaaacaaaccaaccgatgtctgtacgtgggctctgtcagctggaaggtcccggctttcagccgtgatttcccacacccagtacaaggaggatcagttctacagtacttacttctaggtgtactattgttaatggttttaaaatgtaattattgtatttgtaaactgtaccttcattccagtaaggcagttagacacctgagttttagcttttttttccattcctgaaacggatgtaatttaaactgcggtatgtaaatttaatagtagtactgtcgaatggcacaatgcttacagagatacagtgcattttgtcaatatataaaatttaaatataatgttgatagttaccataaagggggtgccacacatcaagaaccttaaatggaaccagaaacaagcaagcaaacaaacaaacaaacaaacaaaaccttacttttcttcactccttattacattttcctctagagctaaagaaacttctagcttcggtttagtgggttaaattcagaaactatttcagaaaaaaaaaaaaattctgaagttacagcatattcaaagagaagcattaattaccacttttttaaaagcttttttttcaaaccgcaaatttcataaaaatgcaaactgtgtaaacagggcctcttatttttataacttgtgtaaaaagggaaaatcaattcatatttaaagtttaagtagtattaaattatatccaagagtgaagaggatgttgaaatcttacctgaccccatgccccttctttgcagtttagcaaatgttgagattgctaaatcatcagattaaagccaacttgatttttaaagtttcaagactttctgaagctgaactggttaaaacttttgcacaattgcttggaacggagggggaggggcctctctggtccagcacaggtaccttgtttcttccctactcacaagaatcaaaacaatgaaagtcaagaaccacagaggggggaaattagttccctgttcagtccaaaaggagaactttaaacttatcatttacgtctttggggaaggaagaaataagctttataagtgaaatcctattcaccttgttgtcctatgaatgttttcggggtgactttaagattcattgtatacatgtgcgagtctctgctattcttggggagttgaaagcagagccaggccagtggccttgaagttcagtaaatgccacagttctggggcaaaggtaggcatgagggttctgcccctcagcacaggaatcagagcagtgtcttgtaaggtctaaagattaagtcttccagtaagccacaagttattttgtaacagagttggggagttttggcactcgctgctgactttcattttgtatccactcaaatggagtcttcaactcttttcaactttagaatcaaattaatttttttttttttttttttttttttacacaaggtttactctgtgtaactgtcctggatgttctggaactctttttgtagaccaggctggcctcgaactcagagagatccacctgcctgtgctccccaagtgctgggattaaaggcgtgtgccaccatgcctggcttagattaaattttttaagtcttacttcaccagtgagattgtgattggcagttgtttcgagagagctttgtagcttaatctatgttctcttcaatcaatgcttgctaccaaaagaatgtccaaaatgatctatttttcctgggaacaattcatctatttaaataggctcttgcctagttccccaaagcagcctgtctttgaaggtttttttgaacaaaataattttttcacaaaaagtttggttttgaaatcaaaatagagaaataaaatgtaaattttaaatctaatggaacatgaggaaatgaaaaaacttaagccaatggagagtaaaagcagaaaaaaatgaaacttacctagaatgtgattatattatgtttttaagtagtcaattcatggaaaaatattgaatattaacacaaagcatattaaaaatatgtaaatattactgtttctcatgtctttctctttatatcttattttatatagttttagaatgaattggtcattaaatacagtgtttctttccaaagaataattttgttgatattgtaaaaatgtaattaaagatagagacttgaatagtctctaacattatccaaatgtttctaggaaccaaattcaaagctgtgaagaaagcttgcaatccctgaattggcttttgtgaaatggaatgacggtgggtaatctcaaaattcagacttgaatagtcagagctgaagtggggaatgggtggttccttctggttcagaaaataggtcaaataacagcatttgctcgcatcagggatggagatgttggtgatgtttggttttactctcgcaggctttcgtctcctgttgaaggtgtatctgtagcccagtgggataagagttcatgttctgagatgtggtcctagacaaggcaggcaaggtttcagtcatcaatacctatcaggtcaggttcccttttgtctatacaaaatgggttagctcatagccagatggtttgcaggacagtgagctaaattaggacaagattctggttagccaaagagctgtttcctaagcactctgatttttttttaaagctgatagaaagtgtaaatgttctattttgacgacatggaaagtatgttttcctcttcaaataaatcccttatttttatgaaattttcaaaaataaattcttgtttaaaatagtctgaatgttatcatagttggaacttggcaattactaatttgaaattctatgagatgtatctccagctaaaatggcaattccctgtatgctatctggggctcagtttacctctaaggaagactgtcagagtgcaaatggttttgagtgacgggaaagtcaaagggcaaatgtttgtgcttttttctttttctgtcttatatacttcttcttggtctcagaatgcaaagtatcagagccatagttacacacatttccacttttaacgcttcttttgaaggaagcagatccacttttgccccgccactcatgcctgctgtgcagactcagacgagtccctgccctcttcacgcctttggggtgagaggggagccatatgtaagtagttttcaagcttttcttaatgggacttttctttttctaataaaatcatgcctggaatcctgtaaagattgttgcctggctgtgaaggggcttctccagatcctgaaatatagcatcacaatacgtaaatgactcccgatggatctcccagctctgaagacttgctcttctacttcacatgtgtagccacgacgatcagctggcacacagtacaattagctgtgtagtgagtgctccccagctatcagtcatgaaacatatcactttgctcaacctgtttttaaaaaagctccaaaatggtaaaaatgcttttcagtctttgttttcccaataatggtattgaggcctaagctgattaacttcccccaaagtggtaccacagctggtaacgaccccaatgatcctgaaaaaaatggaatgagtaccttgctgtttcrtttagttyattttgggaaaataatccatttgaatgtcaagataaaaaggcaccaggaaaagtcctcattggaaggattaaagatgagcctggtaagatgttaagatgtaagatgttaagatgtgttactgtaaaaaaaaaaagctt

SEQ ID NO: 4 - protein, 827 aa, Psammomys obesus
MMEGLKKRTRKAFGIRKKEKDTDSTGSPDRDGMQPSPHELPYHSKAECAREGGNKASKKSNGAPNGFYAEIDWERYNSPELDEEGYSIRPEEPGSTKGKHFYSSSESEEEEESHKKFNIKIKPLQSKDILKNAATVDELKASIGNIALSPSPVRKSPRRSPGAIKRNLSSEEVARPRRSTPTPELTSKKPLDDTLALAPLFGPPLESAFDGHKTEVLLDQPEIWGSGQPVNPSMESPKLARPFPTGTPPPLPPKTVPATPPRTGSPLTVATGNDQAATEAKIEKLPSISDLDSIFGPVLSPKSVAVNTEETWVHFSDASPEHVTPELTPREKVVTPPAASDIPADSPTPGPPGPPGSAGPPGPPGPRNVPSPLNLEEVQKKVAEQTFIKDDYLETLSSPKECGLGQRATPPPPPPPTYRTVVSSPGPGSGSGTGTASGASSPARPATPLVPCSCSTPPPPPPRPPSRPKLPPGKPGVGDVSRPFSPPIHSSSPPPIAPLARAESTSSISSTNSLSAATTPTVENEQASLVWFDRGKFYLTFEGSSRGPSPLTMGAQDTLPVAAAFTETVNAYFKGADPSKCIVKITGEMVLSFPAGITRHFANNPSPAALTFRVINSSRLEHVLPNPQLLCCDNTQNDANTKEFWVNMPNLMTHLKKVSEQKPQATYYNVDMLKYQVSAQGIQSTPLNLAVNWRCEPSSTDLRIDYKYNTDAMSTAVALNNVQFLVPIDGGVTKLQAVLPPAVWNAEQQRILWKIPDISQKSENGGVGSLLARFQLAEGPSKPSPLVVQFTSEGSTLSGCDIELVGAGYGFSLIKKRFAAGKYLADN

SEQ ID NO: 5 - DNA, 366 bp, Psammomys obesus
  misc_feature (305)..(305): n = any nucleotide
tcgcggatccagacgctgcgtttgctggctttgatgaaatttttaatttttcaatatcaggaatgtttaactatgccatgaatttatggtagtcaaggttggaaggcaggggagaggacacagggagtaaaggcacttgcccccaagccttacaacctgaattccatcccagagtgcctaatggttgaaggacggaactgaatatctctagctgtcctctatcctccacagatacacagtgaatgcatcaacgtaaaaaattacagctagaaataatgtcgtgccattcattgtattttacattngtncatcttngnttttccatantaaaaatgtctnagacataccacttaaaaaaaaaagctt

SEQ ID NO: 6 - DNA, 442 bp, Psammomys obesus
ccctatccgctacccctgggagggacacaaaaacacattttgtgtttttgaaaaaactgaggtcaccagactcttgtattgtcttcttggacttctctcaggaacactcaggactctccccacacaacaccgttcttgaaccgttctaacaaatgtttaaagtggtttcctttgaaccacattaaatttagtttaagcagtcaccagtgggctagcagttctgggttgggcagcacatcttgtacaagctcttccatctgccaggatcaccacctctctgacttgcacatttgtgggttccccacagacgaatgggatgagtgaaagagtgagtatgttctgttgggccttcagtaacagaagactgattcagaaagtagcacacgtcacatttttctgtaggttggtttgtttagtttcatttttgatttgtggaacaaaa

SEQ ID NO: 7 - DNA, 189 bp, Psammomys obesus
tgagagtccatctcagcttatttcattgagatgttttgattaagaagtatgactagattaaaaaattctatatagcttggcattgttgatagtttatatatttcactgatttctggtcccttgaaagttacttggtgatcaacatagtgtagtgaaaggattgggatggacattaaaaaaaaaaagctt

SEQ ID NO: 8 - DNA, 378 bp, Psammomys obesus
  misc_feature (4)..(4): n = any nucleotide
tggntactcttgnaagcaccttgaaagttcagcctgctaacttacctttttagctttagattgcttagatattcaaatgagagtgtggtgcaaatcctgattacaaggagtttgagtttggagtgattggcaatgcacttgtgaggctgtgtgcctatggtcctaggacttaggaggcagggatagaaggaccaggtgctgaaagacagccttggctagttagtggacagatacataaatgtactgcatgagattctttcagaataacaacctccttttaaagaagttacttctgacatggaatctgttgcctgcttttggatcactaccccctggtgggacacctttgccagaccatggaggaagaactgtcttgat

SEQ ID NO: 9 - DNA, 342 bp, Psammomys obesus
  misc_feature (27)..(27): n = any nucleotide
aacgtgagctttttggaaggcccaganaatttaaggaaagtgtcccggcaaatcctgattacaaggagtttgagtttggagtgattggcaatgcacttgtgaggctgtgtgcctatggtcctaggacttaggaggcagggatagaaggaccaggtgctgaaagacagccttggctagttagtggacagatacataaatgtactgcatgagattctttcagaataacaacctccttttaaagaagttacttctgacatggaatctgttgcctgcttttggatcactaccccctggtgggacacctttgccagaccatggaggaagaactgtcttgatgggann

SEQ ID NO: 10 - DNA, 19 bp, artificial sequence
  primer
gtagactgcgtaccaattc

SEQ ID NO: 11 - DNA, 19 bp, artificial sequence
  primer
gacgatgagtcctgagtaa

SEQ ID NO: 12 - DNA, 24 bp, artificial sequence
  AGT-119 (set 1) forward primer
ggattatcgtgaccagataggagc

SEQ ID NO: 13 - DNA, 21 bp, artificial sequence
  AGT-119 (set 1) reverse primer
acgcatcttgttgggctaatg

SEQ ID NO: 14 - DNA, 20 bp, artificial sequence
  AGT-119 (set 2) forward primer
cgcaaccaaattagccagtg

SEQ ID NO: 15 - DNA, 23 bp, artificial sequence
  AGT-119 (set 2) reverse primer
gcatcttgttgggctaatgacat

SEQ ID NO: 16 - DNA, 19 bp, artificial sequence
  AGT-120 forward primer
acccagatgctggctgact

SEQ ID NO: 17 - DNA, 22 bp, artificial sequence
  AGT-120 reverse primer
ctggccaccagtcagtgaataa

SEQ ID NO: 18 - DNA, 28 bp, artificial sequence
  AGT-121 forward (insertion) primer
aaaacatgagagaagccatactaattca

SEQ ID NO: 19 - DNA, 31 bp, artificial sequence
  AGT-121 reverse (deletion) sequence
cacaaaacatgagatactaattcataagtga

SEQ ID NO: 20 - DNA, 24 bp, artificial sequence
  AGT-121 reverse primer
tgaaaccaagatagcacaaacgaa

SEQ ID NO: 21 - DNA, 22 bp, artificial sequence
  AGT-122 forward primer
catcccagagtgcctaatggtt

SEQ ID NO: 22 - DNA, 25 bp, artificial sequence
  AGT-122 reverse primer
acgttgatgcattcactgtgtatct

SEQ ID NO: 23 - DNA, 20 bp, artificial sequence
  AGT-422 forward primer
cagtcaccagtgggctagca

SEQ ID NO: 24 - DNA, 20 bp, artificial sequence
  AGT-422 reverse primer
cctggcagatggaagagctt

SEQ ID NO: 25 - DNA, 30 bp, Artificial Sequence
  AGT-123 forward primer
gcttggcattgttgatagtttatatatttc

SEQ ID NO: 26 - DNA, 30 bp, artificial sequence
  AGT-123 reverse primer
cactacactatgttgatcaccaagtaactt

SEQ ID NO: 27 - DNA, 21 bp, artificial sequence
  AGT-504 forward primer
gaatctgttgcctgcttttgg

SEQ ID NO: 28 - DNA, 20 bp, artificial sequence
  AGT-504 reverse primer
ctccatggtctggcaaaggt

SEQ ID NO: 29 - DNA, 22 bp, artificial sequence
  Beta-actin forward primer
gcaaagacctgtatgccaacac

SEQ ID NO: 30 - DNA, 23 bp, artificial sequence
  Beta-actin reverse primer
gccagagcagtgatctctttctg

SEQ ID NO: 31 - DNA, 25 bp, artificial sequence
  Beta-actin probe
tccggtccacaatgcctgggaacat

SEQ ID NO: 32 - DNA, 19 bp, artificial sequence
  Cyclophilin forward primer
cccaccgtgttcttcgaca

SEQ ID NO: 33 - DNA, 20 bp, artificial sequence
  Cyclophilin reverse primer
ccagtgctcagagcacgaaa

SEQ ID NO: 34 - DNA, 23 bp, artificial sequence
  Cyclophilin probe
cgcgtctccttcgagctgtttgc